(19) United States
(12) Patent Application Publication
(10) Pub. No.: US 2023/0042448 A1
(43) Pub. Date: Feb. 9, 2023

(54) METHOD AND BIOMARKER FOR DETECTION OR DIAGNOSIS OF MYOCARDIAL INFARCTION

(71) Applicant: ACADEMIA SINICA, Taipei (TW)

(72) Inventors: Patrick CH HSIEH, Taipei City (TW); Hung-Chih CHEN, Lukang Township (TW)

(21) Appl. No.: 17/806,932

(22) Filed: Jun. 14, 2022

Related U.S. Application Data
(60) Provisional application No. 63/210,188, filed on Jun. 14, 2021.

(51) Int. Cl. C12Q 1/689

(52) U.S. Cl. CPC C12Q 1/689; C12Q 2600/118; C12Q 2600/112; C12Q 1/6869

(57) ABSTRACT

The disclosure provides a method for or the early diagnosis, prognosis and differentiation of myocardial infarction (MI). The method comprises performing genetic analysis on gut microbiota. The disclosure also provides a biomarker and kit for the early diagnosis, prognosis, recurrence and differentiation of MI.

### PRIORITY INFORMATION

This application claims benefit of and priority to U.S. Provisional Patent Application No. 63/210,188, filed on Jun. 14, 2021, the entirety of which is attached herewith.

### SEQUENCE LISTING

The instant application contains a Sequence Listing which is submitted electronically in ASCII format and is hereby incorporated by reference in its entirety. The ASCII copy, created on Sep. 26, 2022, is named “G4590-12700US_Seq_Listing_20220926.txt” and is 6 kilobytes in size.

### FIELD OF THE INVENTION

This invention relates to a field of disease diagnosis and/or detection. More specifically, the invention relates to a method, biomarkers and a kit for the early diagnosis, prognosis, recurrence and differentiation of myocardial infarction (MI).

### BACKGROUND OF THE INVENTION

A myocardial infarction (MI), commonly known as a heart attack, occurs when blood flow decreases or stops to a part of the heart, causing damage to the heart muscle. A number of tests are useful to help with diagnosis, including electrocardiograms (ECGs), blood tests and coronary angiography. MI includes both non-ST-segment elevation myocardial infarction (NSTEMI) and ST-segment elevation myocardial infarction (STEMI). Distinction between NSTEMI and STEMI is vital as treatment strategies are different for these two entities.

An ECG, which is a recording of the heart's electrical activity, may confirm an STEMI, if ST elevation is present. Commonly used blood tests include troponin and, less often, creatine kinase MB. STEMI is the term cardiologists use to describe a classic heart attack. It is one type of myocardial infarction in which a part of the heart muscle (myocardium) has died due to the obstruction of blood supply to the area. STEMI presents with central chest pain that is classically heavy in nature, like a sensation of pressure or squeezing. Examination is variable, and findings range from normal to a critically unwell patient in cardiogenic shock. STEMI is a life-threatening, time-sensitive emergency that must be diagnosed and treated promptly. Immediate and prompt reperfusion can prevent or minimize myocardial damage and improve the chances of survival and recovery.

An enormous body of evidence now demonstrates that the inflammatory responses occurring after MI play critical roles during cardiac repair and are driven by many cell types (Porrello E R et al., Science. 2011; 331:1078-80; Aurora A B et al., J Clin Invest. 2014; 124:1382-92). Apoptotic neutrophils in the injured myocardium stimulate macrophage recruitment and promote the clearance of necrotic debris and apoptotic cells. Ablation of neutrophils in genetic knockout models has been shown to result in worse cardiac function and increased fibrosis after myocardial infarction (MI) (Gamin A J et al., Circulation. 2004; 109:990-6; Nathan C and Ding A. Cell. 2010; 140:871-82). In addition, macrophages derived from recruited monocytes or other similar precursors build inflammatory microenvironments to activate cardiofibroblasts for cardiac remodeling, and endogenous stem cells for heart regeneration, either by cell fusion or transdifferentiation (Wu J M et al., Circ Res. 2015; 116:633-41).

There is still a need to develop a strategy to detect and/or diagnose MI.

### SUMMARY OF THE INVENTION

A method and biomarker for diagnosing MI is established in this present disclosure based on gut microbiota analysis.

The present disclosure provides a method for the early detecting or diagnosing likelihood of myocardial infarction (MI), predicting prognosis or treatment outcome and/or differentiating MI in a subject, wherein the method comprises:
obtaining a sample comprising gut microbiota of the subject;
performing genetic analysis on the gut microbiota; and
determining the likelihood of MI as an indicator of detection or diagnosis of MI or prediction of prognosis or treatment outcome of MI if:
(i) decreased abundance of gut microbial genera Bacteroidetes, and/or Bifidobacterium;
(ii) increased abundance of gut microbial genera Streptococcus, Clostridium and/or Butyricimonas;
(iii) increased abundance ratio of gut microbial genera Firmicutes Bacteroidetes; and/or
(iv) increased complexity and diversity of gut microbial genera;
in the sample is found when compared to a normal control.

In some embodiments of the disclosure, the Bifidobacterium comprises Bifidobacterium adolescentis and/or Bifidobacterium ruminantium; Butyricimonas comprises Butyricimonas virosa; Clostridium comprises Clostridium asparagiforme; Streptococcus comprises Streptococcus parasanguinis and/or Streptococcus salivarius.

In one embodiment, the present disclosure provides a method for the early detecting or diagnosing likelihood of myocardial infarction (MI), predicting prognosis or treatment outcome and/or differentiating MI in a subject, wherein the method comprises:
obtaining a sample comprising gut microbiota of the subject;
performing genetic analysis on the gut microbiota; and
(i) determining the likelihood of MI as an indicator of detection or diagnosis of MI or prediction of prognosis or treatment outcome if a decreased abundance of gut microbial genera Bacteroidetes, and/or Bifidobacterium in the sample is found when compared to a normal control.

In some embodiments of the disclosure, the determination of the above (i) comprises determining the abundance of the gut microbial bacteria Bifidobacterium adolescentis and Bifidobacterium ruminantium.

In another embodiment, the present disclosure provides a method for the early detecting or diagnosing likelihood of myocardial infarction (MI), predicting prognosis or treatment outcome and/or differentiating MI in a subject, wherein the method comprises:
obtaining a sample comprising gut microbiota of the subject;
performing genetic analysis on the gut microbiota; and
(ii) determining the likelihood of MI as an indicator of detection or diagnosis of MI or prediction of prognosis or treatment outcome if increased abundance of gut microbial genera Streptococcus, Clostridium and/or Butyricimonas in the sample is found when compared to a normal control.

In some embodiments of the disclosure, the determination of the above (ii) comprises determining the abundance of the gut microbial bacteria Butyricimonas virosa, Clostridium asparagiforme, Streptococcus parasanguinis and Streptococcus salivarius.

In another embodiment, the present disclosure provides a method for the early detecting or diagnosing likelihood of myocardial infarction (MI), predicting prognosis or treatment outcome and/or differentiating MI in a subject, wherein the method comprises:
obtaining a sample comprising gut microbiota of the subject;
performing genetic analysis on the gut microbiota; and
(iii) determining the likelihood of MI as an indicator of detection or diagnosis of MI or prediction of prognosis or treatment outcome if an increased abundance ratio of gut microbial genera Firmicutes/Bacteroidetes in the sample is found when compared to a normal control.

In some embodiments of the disclosure, the method comprises determining the abundance of the gut microbial genera Firmicutes and Bacteroidetes.

In another embodiment, the present disclosure provides a method for the early detecting or diagnosing likelihood of myocardial infarction (MI), predicting prognosis or treatment outcome and/or differentiating MI in a subject, wherein the method comprises:
obtaining a sample comprising gut microbiota of the subject;
performing genetic analysis on the gut microbiota; and
(iv) determining the likelihood of MI as an indicator of detection or diagnosis of MI or prediction of prognosis or treatment outcome if an increase of the complexity and diversity of gut microbial genera in the sample is found when compared to a normal control.

In some embodiments of the disclosure, the sample described herein comprises a tissue of the gut. Preferably, the sample is a fecal sample.

In some embodiments of the disclosure, the determination of the above (i), (ii), (iii) and/or (iv) comprises analyzing 16S rRNA of the metagenome of the gut microbiota; preferably, the method comprises analyzing V3-V4 region of the 16S rRNA of the metagenome of the gut microbiota. In one preferred embodiment of the disclosure, the determination of the above (i), (ii), (iii) and/or (iv) comprises performing next generation sequencing of 16S rRNA of the metagenome of the gut microbiota.

In some embodiments of the disclosure, the determination of the above (i), (ii), (iii) and/or (iv) comprises performing shotgun metagenomic sequencing the gut microbiota.

In some embodiments of the disclosure, the determination of the above (i), (ii), (iii) and/or (iv) comprises performing linear discriminant analysis (LDA) on the gut microbiota.

In some embodiments of the disclosure, the determination of the above (i), (ii), (iii) and/or (iv) comprises performing effect size (LEfSe) analysis on the gut microbiota.

In some embodiments of the disclosure, the method comprises determining the complexity and diversity of the gut microbiota. In one preferred embodiment of the disclosure, the determination of the above (iv) comprises performing alpha or beta diversity calculation for determining the complexity and diversity of the gut microbiota. Preferably, the complexity and diversity is represented as Shannon index, Chao index or Unweight uniFrac index.

In some embodiments of the disclosure, in (i), the normal control is a level of gut microbial genera Bacteroidetes, and/or Bifidobacterium in gut microbiota of a group of normal subjects.

In some embodiments of the disclosure, in (ii), the normal control is a level of gut microbial genera Streptococcus, Clostridium and/or Butyricimonas in gut microbiota of a group of normal subjects.

In some embodiments of the disclosure, in (iii), the normal control is a ratio of the level of gut microbial genera Firmicutes/Bacteroidetes in gut microbiota of a group of normal subjects.

In some embodiments of the disclosure, in (iv), the normal control is the complexity and diversity in gut microbiota of a group of normal subjects.

In some embodiments of the disclosure, the normal control is obtained by:
obtaining a group of control samples comprising gut microbiota of a group of normal subjects; and
performing genetic analysis on the gut microbiota of the group of normal subjects, wherein the genetic analysis on the gut microbiota of the group of normal subjects is the same as that of the subject.

The present disclosure also provides a biomarker for the early diagnosis, prognosis and recurrence, differentiation of myocardial infarction in a subject, wherein the biomarker is selected from one or more gut microbial genera Bacteroidetes, Bifidobacterium, Streptococcus, Clostridium, Butyricimonas, Firmicutes and Bacteroidetes.

In some embodiment, the biomarker is selected from the group consisting of:
(i) a combination of gut microbial genera Bacteroidetes, and/or Bifidobacterium;
(ii) a combination of gut microbial genera Streptococcus, Clostridium and/or Butyricimonas; and/or
(iii) a combination of gut microbial genera Firmicutes and Bacteroidetes.

The embodiments of the gut microbial genera are those described herein.

The present disclosure also provides a kit for the early diagnosis, prognosis, recurrence and differentiation of myocardial infarction in a subject, wherein the kit comprises a detecting molecule for detecting the biomarker as described herein.

In some embodiments of the disclosure, the detecting molecule is for analyzing 16S rRNA of the metagenome of the gut microbiota. Examples of the detecting molecule include, but are not limited to, a nucleic acid molecule or a polypeptide molecule. Preferably, the detecting molecule is a nucleic acid molecule, such as an oligonucleotide molecule. Examples of the oligonucleotide molecule include, but are not limited to, a primer for specifically amplifying the 16S rRNA region or a probe for specifically hybridizing the 16S rRNA region.

In some embodiments of the disclosure, the detecting molecule is for analyzing V3-V4 region of the 16S rRNA of the metagenome of the gut microbiota.

In some embodiments of the disclosure, the kit further comprises an agent for sequencing. Examples of the agent include, but are not limited to, an agent for next generation sequencing or an agent for shotgun metagenomic sequencing.

In some embodiments of the disclosure, the kit further comprises a group of control samples comprising gut microbiota of a group of normal subjects.

In some embodiments of the disclosure, the MI described herein is STEMI.

The present disclosure also provides a method for treating and/or ameliorating myocardial infarction in a subject, wherein the method comprises colonizing Bifidobacterium adolescentis, Butyricimonas virosa and/or Streptococcus parasanguinis in the gut of the subject.

In some embodiments of the disclosure, the method further comprises colonizing a butyrate-producing bacterium in the gut. Examples of the butyrate-producing bacterium include, but are not limited to, Anaerotruncus colihominis, Bacteroides caccae, Bacteroides thetaiotaomicron, Clostridium symbiosum, Collinsella aerofaciens, Coprococcus comes, Providencia stuartii, and Ruminococcus torques.

In some embodiments of the disclosure, the method is for improving cardiac function, improving preservation of cardiac mechanical properties, and/or decreasing average infarct size.

The present disclosure is described in detail in the following sections. Other characteristics, purposes and advantages of the present invention can be found in the detailed description and claims.

### BRIEF DESCRIPTION OF THE DRAWINGS

FIGS. 1A to 1P shows enrichment of butyrate-producing gut microbiome after cardiac injury. FIG. 1A shows a schematic illustration of the experimental design. Stools from ST-elevation myocardial infarction (STEMI) patients were collected right after percutaneous intervention (PCI) (STEMI-T1) and on day 28 after PCI (STEMI-T2). Stool samples from both controls (Ctrl) and STEMI patients were subjected to 16s rRNA V3-V4 NGS (FIGS. 1B-1G) and metagenome shotgun (FIGS. 10H-10I). FIG. 1B shows Venn diagram showing the overlapping of operational taxonomic unit (OTU) as named by Ctrl, STEMI-T1 and STEMI-T2. FIG. 1C shows Shannon diversity index of Ctrl and STMEI gut microbiota. FIG. 1D shows unweighted Unifrac distance measurement of the gut microbiota in Ctrl and STEMI samples. FIG. 1E shows the Firmicutes/Bacteroidetes ratio of gut microbiota in Ctrl and STEMI samples. FIG. 1F shows Spearman correlation of cardiac ejection fraction (EF, %) with Shannon's index (upper panel) and Pielou's evenness (lower panel). Blue dots represent non-diabetic patients, and red dots represent diabetic patients. FIG. 1G shows differentially abundant bacterial genera in Ctrl and STEMI samples. FIG. 1H shows linear discriminant analysis (LDA) scores computed with features distinct from Ctrl and STEMIT1 in the species level via metagenome shotgun analysis of 5 Ctrl and 11 STEMIT1 samples. FIG. 11 shows that an abundance of Ba., Br., By., Sp. and Ss. was confirmed with quantitative PCR (qPCR). FIG. 1J shows an illustration of the experimental design for a nonhuman primate disease model. The monkeys were subjected to cardiac ischemic/reperfusion (IR) injury for ninety minutes. The stool samples were collected at pre-IR, IR day 1 (IRD1), IR day 7 (IRD7) and IR day 28 (IRD28) and subjected to 16S rRNA V3-V4 NGS. FIG. 1K shows Venn diagram showing the overlapping of operational taxonomic unit (OTU) in nonhuman primate stools at IR, IRD1, IRD7 and IRD28. FIG. 1L shows Shannon diversity index of nonhuman primate gut microbiota in response to IR. FIG. 1M shows unweighted Unifrac distance measurement of the gut microbiota in nonhuman primate stool samples. FIG. 1N shows the Firmicutes/Bacteroidetes ratio of gut microbiota in nonhuman primate stool samples in response to IR. FIG. 10 shows linear discriminant analysis (LDA) scores (left panel) and abundance (right panel) computed with distinct features in nonhuman primate gut microbiota in response to IR. FIG. 1P shows qPCR confirmation of Ba., By., Sp., Ss. and Sv. abundance in nonhuman primate stool samples. Data in FIGS. 10C-10E, 10I, 10L-10N and 10P were analyzed with Kruskal-Wallis test and are represented as mean±SEM. *p<0.05, **p<0.01, ***p<0.001, ****p<0.0001. Bifidobacterium adolescentis (Ba.); Bifidobacterium ruminantium (Br.); Butyricimonas virosa (By.); Streptococcus parasanguinis (Sp.); Streptooccus salivarius (Ss.); Subdoligranulum variabile (Sv.).

FIGS. 2A to 2F shows microbiome-driven precision diagnostics for STEMI using machine learning. FIG. 2A shows workflow for supervised machine learning. FIG. 2B shows respective sample size for training and test sets. FIG. 2C shows a model trained with different algorithms on a training dataset for the diagnosis of myocardial infarction. FIG. 2D shows a confusion matrix of the test set. FIG. 2E shows receiver operating characteristic (ROC) curve performance using Ctrl predominant bacteria Bifidobacterium adolescentis (Ba.) and Bifidobacterium ruminantium (Br.). FIG. 2F shows ROC curve performance using STEMI predominant bacteria include Streptococcus parasanguinis (Sp.), Streptooccus salivarius (Ss.) and Butyricimonas virosa (Bv.).

FIGS. 3A to 3J show that STEMI fecal microbiota transplantation (FMT) in germ free (GF) mice deteriorates post-injury cardiac repair. 12 week-old germ free (GF) mice were colonized with human fecal microbiota for 7 days before cardiac injury. FIG. 3A shows experimental design for a myocardial infarction (MI) model on FMT-GF mice (upper panel), and the survival rate of FMT-GF mice subjected to MI for 21 days (lower panel). FIG. 3B shows qPCR-based determination of fecal bacterial load for the FMT-GF mice subjected to MI. FIG. 3C shows experimental design for angiotensin II (AngII) challenge on FMT-GF mice (upper panel), and the survival curve of FMT-GF mice subjected to AngII challenge for 14 days (lower panel). FIG. 3D shows the fecal bacterial load for the FMT-GF mice administered with AngII. FIG. 3E shows differential grouping of the gut microbiota in Ctrl and STEMI samples in the unweighted principal coordinates analysis (PCoA). FIG. 3F shows the ratio of gut Firmicutes relative to Bacteroidetes in FMT mice after cardiac injury. FIG. 3G shows echocardiographic analysis of left ventricular ejection fraction (EF, %) and fraction shortening (FS, %) in FMT-GF mice on day 14 after AngII challenge. FIG. 3H shows changes of left ventricular end-diastolic volume (EDV) and end-systolic volume (ESV) of FMT-GF mice on day 14 after AngII challenge. FIG. 3I shows cardiac catheterization parameters of FMT mice on day 14 after AngII challenge, including end-systolic pressure volume relationship (ESPVR), end-diastolic pressure volume relationship (EDPVR), preload recruitable stroke work (PRSW) and ratio of maximal left ventricular pressure rise (dP/dtₘₐₓ) to end-diastolic volume (EDV). FIG. 3J shows changes in the size of cardiomyocytes in FMT mice subjected to AngII challenge for 14 days. The illustration of muscle orientation in the heart is shown in the upper left panel. The representative immunofluorescent staining of cardiac tissues with WGA-488 co-stained with actinin is shown in the upper right panel. The statistical analysis of cardiomyocyte size is shown in the lower panel with the cell number listed in the inset of each bar. Kruskal-Wallis test was used to analyze data in FIGS. 3B, 3D and 3G-3J; two-way ANOVA was used to analyze data in FIG. 3F. Data are represented as mean±SEM. *p<0.05, **p<0.01, ***p<0.001, ****p<0.0001.

FIGS. 4A to 41 show ketone body biosynthesis and degradation pathways are enriched in STEMI patients. FIG. 4A shows enrichment of metabolic pathways revealed by shotgun metagenomics with Ctrl and STEMI-T1 samples. FIG. 4B shows a schematic illustration of human plasma metabolomics profiling using nuclear magnetic resonance (NMR) and liquid chromatography-mass spectrometry (LC-MS). FIG. 4C shows PCoA grouping of Ctrl, STEMI-T1 and STEMI-T2 based on NMR plasma metabolite profiling. FIG. 4D shows metabolic pathway enrichment in the Ctrl, STEMI-T1 and STEMI-T2 plasma samples via LC-MS analysis. Enrichment Ratio is computed by observed hits/expected hits. FIG. 4E shows ROC curve for STEMI prediction based on the enrichment of lactate, pyruvate, acetone, acetoacetate, glutamate, 3-hydroxybutyrate and butyrate. FIG. 4F shows the Level of human plasma β-hydroxybutyrate using colorimetric assay. FIG. 4G shows Nonhuman primates subjected to cardiac ischemic/reperfusion (IR) injury for ninety minutes and the plasma samples were collected at −1D (pre-IR), D1 (IRD1), D7 (IRD7) and D28 (IRD28) and subjected to LC-MS metabolomics profiling. FIG. 4H shows enrichment of the metabolic pathways of Macaque rhesus at pre-IR, IRD1, IRD7 and IRD28 using LC-MS. FIG. 41 shows the Level of Macaque rhesus plasma β-hydroxybutyrate using colorimetric assay. Data in FIGS. 4F and 4I were analyzed with Kruskal-Wallis test and are represented as mean±SEM. *p<0.05, **p<0.01.

FIGS. 5A to 5H show that butyrate supplementation confers better preservation of cardiac function after myocardial injury. FIG. 5A shows experimental design for the effects of butyrate on post-MI cardiac repair (upper panel) in SPF mice. Half of the mice were also treated with antibiotics (ABX) to deplete the gut microbiota. The relative bacterial load in feces on MI day 21 was determined with 16S rRNA qPCR (lower panel). FIG. 5B shows that level of fecal butyrate on MI day 21 using HPLC analysis. FIG. 5C shows that plasma level of β-hydroxybutyrate on MI day 21 using colorimetric assay. FIGS. 5D-5E show changes of the left ventricular EF (FIG. 5D) and FS (FIG. 5E) in response to butyrate supplementation on MI day 21, and the survival rate of SPF mice after 21 days post-MI. FIGS. 5F-5G show representative images of cardiac infarct size labeled with picrosirus red (FIG. 5F) and statistics (FIG. 5G). FIG. 5H shows catheterization analysis of post-MI cardiac function, including ESPVR, EDPVR, PRSW and dP/dtₘₐₓ (vs EDV). Data were analyzed with Kruskal-Wallis test and are represented as mean±SEM. *p<0.05, **p<0.01, ***p<0.001, ****p<0.0001.

FIGS. 6A to 6K show that inoculation of B. adolescentis and B. virosa improves post-MI cardiac function in germ-free mice. FIG. 6A shows that shotgun metagenomics analysis revealed the enrichment of ketogenesis in STEMI-T1 samples. Abundance of genes encoding ketogenic enzymes in Ctrl and STEMI samples was presented in the bar charts. FIG. 6B shows butyrate production capabilities of Ba., By. and Sp. FIG. 6C shows β-hydroxybutyrate production capabilities of Ba., By. and Sp. FIG. 6D shows experimental design of the myocardial infarction (MI) model in gnotobiotic mice inoculated with Ba., By., and Sp. (upper panel), and the survival curve of gnotobioic mice subjected to MI for 21 days (lower panel). FIG. 6E shows changes of bacterial load in gnotobiotic mice determined with 16s rRNA qPCR. FIG. 6F shows bacterial load of Ba., By. and Sp. in gnotobiotic mice. FIG. 6G shows echocardiographic analysis of changes in left ventricular ejection fraction (EF, %) and fraction shortening (FS, %) in gnotobiotic mice on MI day 21. FIG. 6H shows post-MI cardiac function analysis in gnotobiotic mice, evaluating ESPVR, EDPVR, PRSW and dP/dtₘₐₓ (vs EDV) using cardiac catheterization. FIG. 6I shows representative histology of cardiac infarct size (left panel) and statistics (right panel) in gnotobiotic mice on MI day 21. Cardiac tissue was stained with picrosirius red to label fibrosis. FIG. 6J shows colorimetric analysis of the plasma level of β-hydroxybutyrate in gnotobiotic mice on MI day 21. FIG. 6K shows representative images of gut in gnotobiotic mice (left panel), intestinal length (upper right panel) and the length of colon (lower right panel) on MI day 21. Data were analyzed with Kruskal-Wallis test and are represented as mean±SEM. *p<0.05, **p<0.01, ***p<0.001, ****p<0.0001. ACAT, Acetyl-CoA acetyltransferase; HMGCS, 3-hydroxy-3-methylglutaryl-CoA synthase; HMGCL, 3-hydroxy-3-methylglutaryl-coenzyme A (HMG-CoA) 2 lyase; OXCT, succinyl-CoA:3-oxoacid CoA transferase (OXCT); ADC, acetoacetate decarboxylase; BDH, beta-hydroxybutyrate dehydrogenase; Bifidobacterium adolescentis (Ba.); Bifidobacterium ruminantium (Br.); Butyricimonas virosa (Bv.); Streptococcus parasanguinis (Sp.); Streptooccus salivarius (Ss.).

### DETAILED DESCRIPTION OF THE INVENTION

The present invention can be more readily understood by reference to the following detailed description of various embodiments of the invention, the examples, and the chemical drawings and tables with their relevant descriptions. It is to be understood that unless otherwise specifically indicated by the claims, the invention is not limited to specific preparation methods, carriers or formulations, or to particular modes of formulating the compounds of the invention into products or compositions intended for topical, oral or parenteral administration, because as one of ordinary skill in the relevant arts is well aware, such things can, of course, vary. It is also to be understood that the terminology used herein is for the purpose of describing particular embodiments only and is not intended to be limiting.

As utilized in accordance with the present disclosure, the following terms, unless otherwise indicated, shall be understood to have the following meaning:

As used herein, the use of “or” means “and/or” unless stated otherwise. In the context of a multiple dependent claim, the use of “or” refers back to more than one preceding independent or dependent claim in the alternative only.

It must be noted that, as used in the specification and the appended claims, the singular forms “a,” “an” and “the” include plural referents unless the context clearly dictates otherwise. Thus, unless otherwise required by context, singular terms shall include the plural and plural terms shall include the singular.

The term “diagnosis” as used herein refers to methods by which the skilled artisan can estimate and/or determine the probability (“a likelihood”) of whether or not a patient is suffering from a given disease or condition. In the case of the present disclosure, “diagnosis” includes using the results of an assay, most preferably a biomarker of the present disclosure, optionally together with other clinical characteristics, to arrive at a diagnosis (that is, the occurrence or nonoccurrence) of MI for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather uses test results together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

The term “early diagnosis” is used herein denotes the screening or testing of individuals to identify a condition before symptoms of the disease or condition appear, for example in healthy individuals, or before the condition is diagnosed or able to be diagnosed using current diagnostic methods.

Similarly, a prognostic risk signals a probability (“a likelihood”) that a given course or outcome will occur. A level or a change in the level of a prognostic indicator, which in turn is associated with an increased probability of morbidity (e.g., worsening renal function, future ARF, or death) is referred to as being “indicative of an increased likelihood” of an adverse outcome in a patient.

The term “subject” as used herein denotes any animal, preferably a mammal, and more preferably a human. Examples of subjects include humans, non-human primates, rodents, guinea pigs, rabbits, sheep, pigs, goats, cows, horses, dogs and cats. According to an embodiment of the present disclosure, the subject is suspected of having MI.

As used herein, the term “sample” refers to a sample obtained from a human or animal subject, preferably, of which metagenomics of gut microorganisms is to be detected. In some embodiments of the disclosure, the sample comprises a tissue of the gut.

As used herein, the term “gut microbiota” refers to the collection of bacteria, yeast, fungi, viruses, protozoans, and the like colonizing the GI tract.

As used herein, the term “genetic analysis” is the analysis of a collection of genetic material (genomes) from a mixed community of organisms.

The phrase “increased complexity” when used herein means increase of the complexity based on taxonomic classification of the gut microbial genera in the gut microbiota of the sample, and/or increased complexity based on the proportional number of gut microbial genera by classification in the gut microbiota of the sample, which may be generally calculated from the amount of 16S rRNA with sequences specific to a particular genus, species, or strain normalized against the total number of sequences in the sample.

As used herein, the term “abundance” refers to the relative representation of a species in a particular ecosystem. It is usually measured as the number or level of individuals.

As used herein, the term “diversity” refers to the variability among the gut microbiota and the ecological complexes of which they are part; this includes diversity within species, between species and of ecosystems.

As used herein, the term “normal control” refers to the profile of gut microbiota of a control population wherein members do not have MI.

The terms “treatment,” “treating,” and “treat” generally refer to obtaining a desired pharmacological and/or physiological effect. The effect may be preventive in terms of completely or partially preventing a disease, disorder, or symptom thereof, and may be therapeutic in terms of a partial or complete cure for a disease, disorder, and/or symptoms attributed thereto. “Treatment” used herein covers any treatment of a disease in a mammal, preferably a human, and includes (1) suppressing development of a disease, disorder, or symptom thereof in a subject or (2) relieving or ameliorating the disease, disorder, or symptom thereof in a subject.

As used herein, the term “colonization” refers to the colonization of an environment, e.g., the gut, intestine or colon, by a microbe, e.g., a bacterium, such that the viable population of that microbe continues over time. A stably colonizing population will generally remain substantially static once colonization is complete, e.g., logarithmically transformed colony forming units associated with the gut will remain in the same quartile after the initial period of colonization when followed within the lifespan of the gut epithelial cells.

An astounding number and diversity of microbes are present at any given moment throughout our bodies. Through co-evolution, these microbes and their animal hosts have developed a mutualistic relationship in which their biological interaction has become essential for survival. Recent studies have shown that gut microbiota can influence the composition, migration and function of various immune cell subpopulations. With different members of the gut microbiota capable of affecting host immune homeostasis in different ways, the heterogeneity of this community may be the basis of individual differences in host immune response (Thaiss C A et al., Nature. 2016; 535:65-74; Honda K and Littman D R. Nature. 2016; 535:75-84).

As an important immune modulator, gut microbiota may have an impact on the efficiency of repair after MI. A recent study has shown strong evidence that gut microbiota play an essential role in effective cardiac repair, and that this may be through the production of specific SCFAs that can modulate the immune system and therefore the inflammatory microenvironment after MI (Tang T W H, et al., Circulation. 2019; 139:647-659). Given the integral role of the immune system during wound repair, gut microbiota have been implicated in the repair of a variety of tissues such as the gastrointestinal tract (Maloy K J and Powrie F. Nature. 2011; 474:298-306) and liver (Cornell R P et al., Hepatology. 1990; 11:916-22), as well as surfaces exposed to the external environment such as skin (Zhang M et al., Microb Ecol. 2015; 69:415-21). Interestingly, recent reports have highlighted a relationship between gut microbiota and severity of myocardial infarction, which remains the leading cause of mortality across all industrialized nations with 1 million Americans estimated to suffer a new or recurrent MI each year (Benjamin E J et al., Circulation. 2017;135:e146-e603). ST-elevation myocardial infarction (STEMI) is a cause of the acute myocardial infarction (AMI), and the recurrence rate and death risk of STEMI remain high (Yeh R W et al., N Engl J Med. 2010; 362:2155-65; Bradley S M et al., JAMA Netw Open. 2019;2:e187348). However, the exact role that gut microbiota play and the mechanisms behind their involvement in effective endogenous cardiac repair after MI remain unclear and have yet to be discovered.

Accordingly, the present disclosure provides methods for the detecting or diagnosing likelihood of myocardial infarction (MI), predicting prognosis or treatment outcome and/or differentiating MI in a subject by determining the abundance of the gut microbial genera as an indicator for the detection, diagnosis and prediction. Moreover, these gut microbial genera can be used as biomarkers of detection, diagnosis and prediction of MI.

Next-generation sequencing (NGS), such as metagenomics and metatranscriptomics, is well suited for examining the microbiota composition and predicting potential functions. This information, therefore, does not provide data on changes in the function of the microbiota, which are needed to understand how the alterations in composition impact function and if it is impacted in a physiologically meaningful way.

In another embodiments of the disclosure, the genetic analysis may involve shotgun sequencing. Shotgun sequencing is a laboratory technique for determining the sequence of a metagenome. The method involves breaking the genome into a collection of small fragments that are sequenced individually. In some embodiments, the genetic analysis may involve 16S-based sequencing. In some embodiments, the method comprises analyzing 16S rRNA of the metagenome of the gut microbiota; preferably, the method comprises analyzing V3-V4 region of the 16S rRNA of the metagenome of the gut microbiota. In one preferred embodiment of the disclosure, the method comprises performing next generation sequencing of 16S rRNA of the metagenome of the gut microbiota.

In one embodiment of the disclosure, the method comprises performing linear discriminant analysis (LDA) on the gut microbiota.

In one embodiment of the disclosure, the method comprises performing effect size (LEfSe) analysis on the gut microbiota.

According to an embodiment of the present disclosure, examples of the sample include, but are not limited to, a rectal swab sample, a fecal sample, or a urine sample from a human or an animal. The sample may also include environmental samples from which a nucleic acid of a gut microorganism may be detected, for example, samples collected from toilet bowls, towels, toilet paper, and the like, with which a human or an animal has come into contact.

In one embodiment of the disclosure, the indicator of detection or diagnosis of MI (preferably STEMI) or prediction of prognosis or treatment outcome of MI (preferably STEMI) according to the disclosure includes: (i) a decrease of abundance of gut microbial genera Bacteroidetes, and/or Bifidobacterium in gut microbiota of the subject when compared to a normal control. Accordingly, the method comprises determining the abundance of the gut microbial genera Bacteroidetes, and/or Bifidobacterium. Examples of gut microbial genera Bifidobacterium include, but are not limited to, Bifidobacterium adolescentis and Bifidobacterium ruminantium.

In one embodiment of the disclosure, the indicator of detection or diagnosis of MI (preferably STEMI) or prediction of prognosis or treatment outcome of MI (preferably STEMI) according to the disclosure includes: (ii) increased abundance of gut microbial genera Streptococcus, Clostridium and/or Butyricimonas in gut microbiota of the subject when compared to a normal control. Accordingly, the method comprises determining the abundance of the gut microbial genera Streptococcus, Clostridium and/or Butyricimonas. Examples of gut microbial genera Streptococcus include, but are not limited to, Streptococcus parasanguinis and Streptococcus salivarius. Examples of gut microbial genera Clostridium include, but are not limited to, Clostridium asparagiforme. Examples of gut microbial genera Butyricimonas include, but are not limited to, Butyricimonas virosa.

In one embodiment of the disclosure, the indicator of detection or diagnosis of MI (preferably STEMI) or prediction of prognosis or treatment outcome of MI (preferably STEMI) according to the disclosure includes: (iii) increased abundance ratio of gut microbial genera Firmicutes/Bacteroidetes in gut microbiota of the subject when compared to a normal control. Accordingly, the method comprises determining the abundance of the gut microbial genera Firmicutes and Bacteroidetes.

In one embodiment of the disclosure, the biomarker according to the disclosure includes: (iv) increased complexity and diversity in gut microbiota of the subject when compared to a normal control. Accordingly, the method comprises determining the complexity and diversity of the gut microbiota. In one preferred embodiment of the disclosure, the method comprises performing alpha or beta diversity calculation for determining the complexity and diversity of the gut microbiota. Preferably, the complexity and diversity is represented as Shannon index, Chao index or Unweight uniFrac index.

In some embodiments of the disclosure, in (i), the normal control is a level of gut microbial genera Bacteroidetes, and/or Bifidobacterium in gut microbiota of a group of normal subjects.

In some embodiments of the disclosure, in (ii), the normal control is a level of gut microbial genera Streptococcus, Clostridium and/or Butyricimonas in gut microbiota of a group of normal subjects.

In some embodiments of the disclosure, in (iii), the normal control is a ratio of the level of gut microbial genera Firmicutes/Bacteroidetes in gut microbiota of a group of normal subjects.

In some embodiments of the disclosure, in (iv), the normal control is the complexity and diversity in gut microbiota of a group of normal subjects.

In some embodiments of the disclosure, the normal control is obtained by:
obtaining a group of control samples comprising gut microbiota of a group of normal subjects; and
performing genetic analysis on the gut microbiota of the group of normal subjects, wherein the genetic analysis on the gut microbiota of the group of normal subjects is the same as that of the subject.

The present disclosure also provides a kit for the early diagnosis, prognosis, recurrence and differentiation of myocardial infarction in a subject, wherein the kit comprises a detecting molecule for detecting the biomarker as described herein, preferably for analyzing of the metagenome of the gut microbiota.

The detecting molecule described herein is used for detecting species and/or amounts of the biomarker. 16S rRNA, and preferably the V3-V4 region of the 16S rRNA region, is regarded as a specific region for taxonomic classification of microbes. Thus, a nucleic acid molecule, preferably an oligonucleotide molecule as a primer for specifically amplifying the 16S rRNA region, or a probe for specifically hybridizing the 16S rRNA region is applied as the detecting molecule.

The kit can further comprise several agents for performing the method for the early detecting or diagnosing likelihood of MI (preferably STEMI), predicting prognosis or treatment outcome and/or differentiating MI (preferably STEMI) as described herein. The agents may be an agent for sequencing such as an agent for next generation sequencing or an agent for shotgun metagenomic sequencing.

Preferably, for providing the normal control, the kit further comprises a group of control samples comprising gut microbiota of a group of normal subjects.

The present disclosure also provides a method for treating and/or ameliorating myocardial infarction in a subject, wherein the method comprises colonizing Bifidobacterium adolescentis (Ba), Butyricimonas virosa (By) and/or Streptococcus parasanguinis (Sp) in the gut of the subject.

In some embodiments of the disclosure, successful colonization with Ba, By and/or Sp shows improved cardiac function in a subject. Furthermore, colonization with Ba, By and/or Sp, shows positive changes in EF and FS; higher ESPVR, PRSW and dP/dt max (vs. EDV) as well as lower EDPVR, revealing improved preservation of cardiac mechanical properties. Moreover, the average infarct size is smaller in the subject with colonization with Ba, By and/or Sp. The subject with colonization with Ba, By and/or Sp also shows an increase in the plasma level of β-hydroxybutyrate. Additionally, the subject with colonization with Ba, By and/or Sp displays a longer intestine and colon. These data demonstrate the cardiac protective role of B. adolesenctis and B. virosa/S. parasangunis and also indicate the contribution of bacteria-associated ketone body metabolism in post-MI cardiac protection.

In some embodiments of the disclosure, the method further comprises colonizing a butyrate-producing bacterium in the gut. Examples of the butyrate-producing bacterium include, but are not limited to, Anaerotruncus colihominis, Bacteroides caccae, Bacteroides thetaiotaomicron, Clostridium symbiosum, Collinsella aerofaciens, Coprococcus comes, Providencia stuartii and Ruminococcus torques.

In some embodiments of the disclosure, the method is for improving cardiac function, improving preservation of cardiac mechanical properties, and/or decreasing average infarct size.

The following examples are provided to aid those skilled in the art in practicing the present invention.

### EXAMPLES

### Materials and Methods:

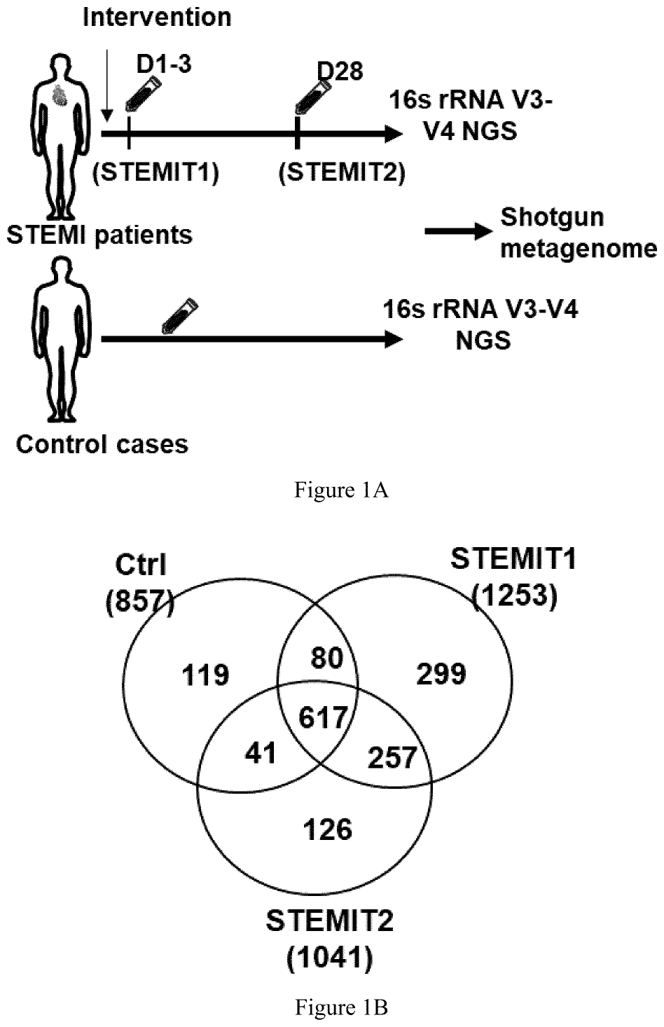

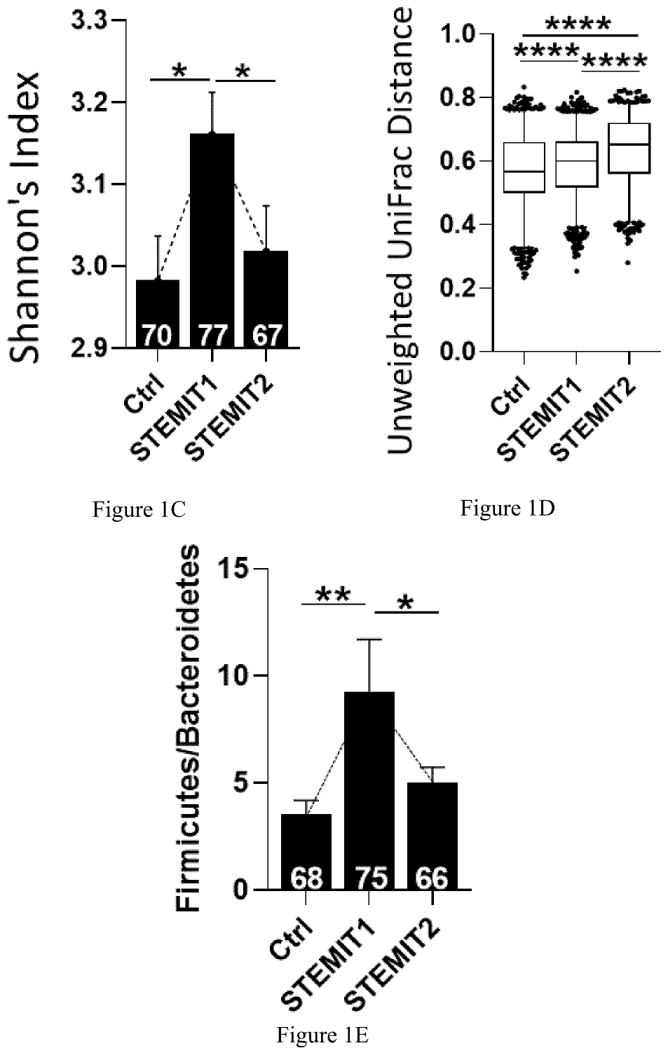

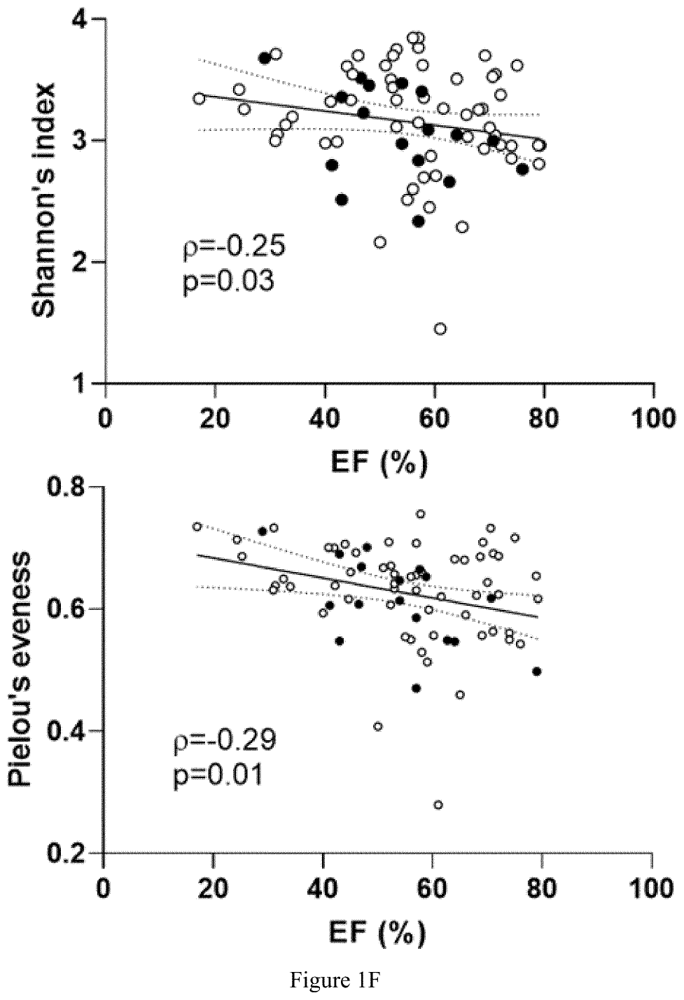

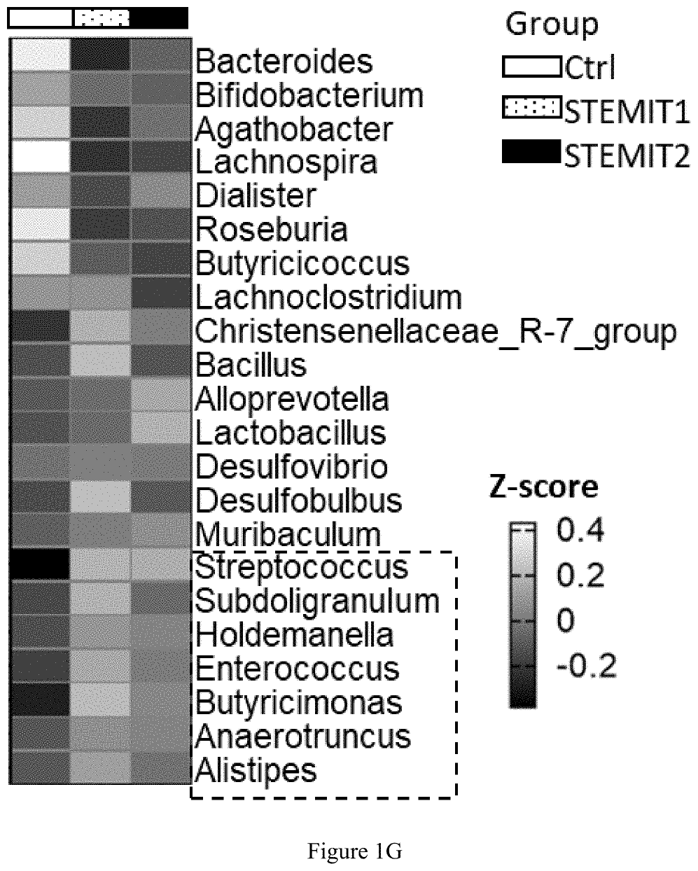

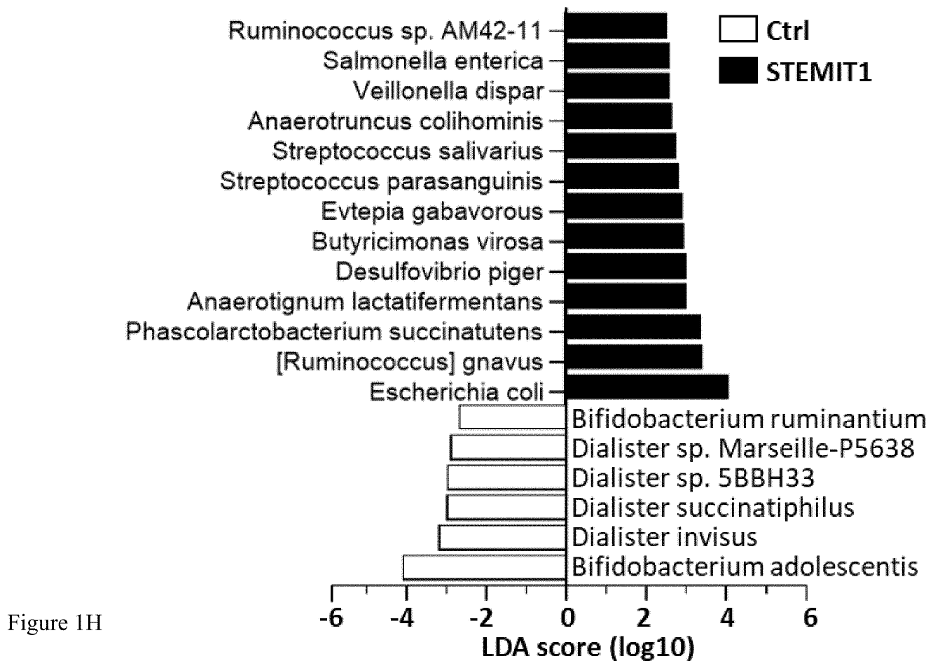

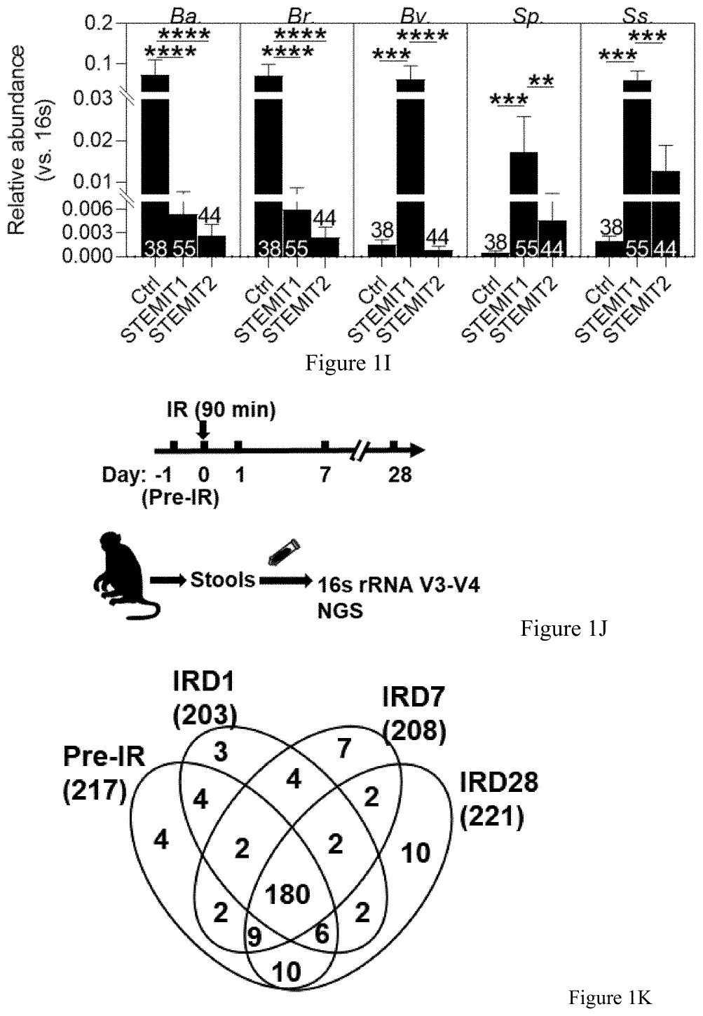

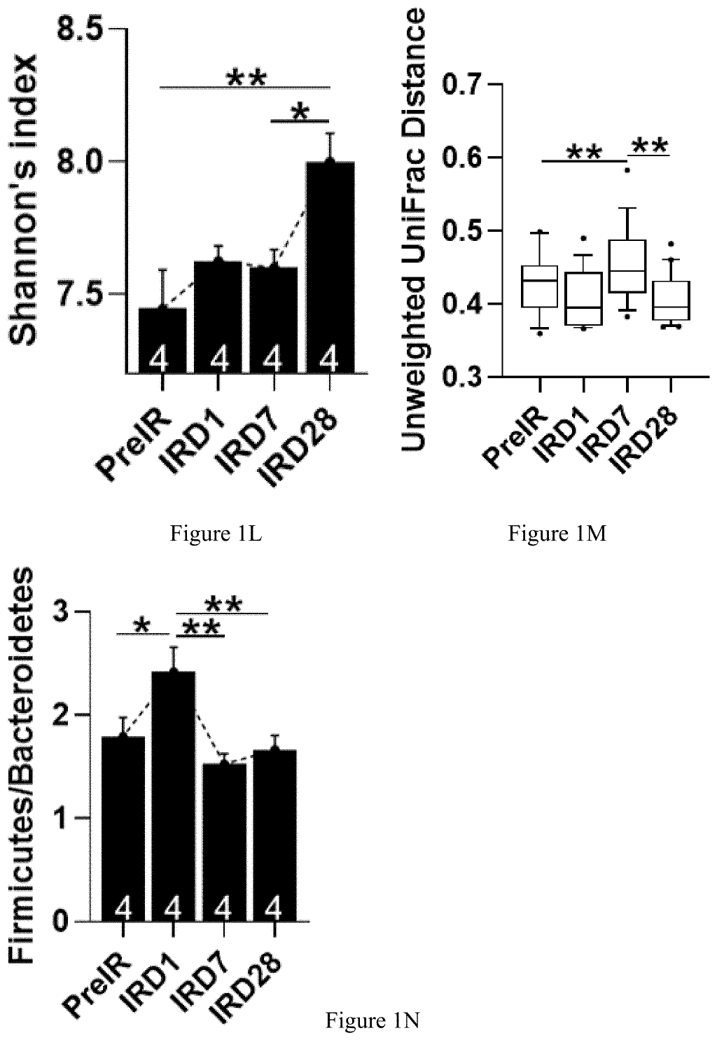

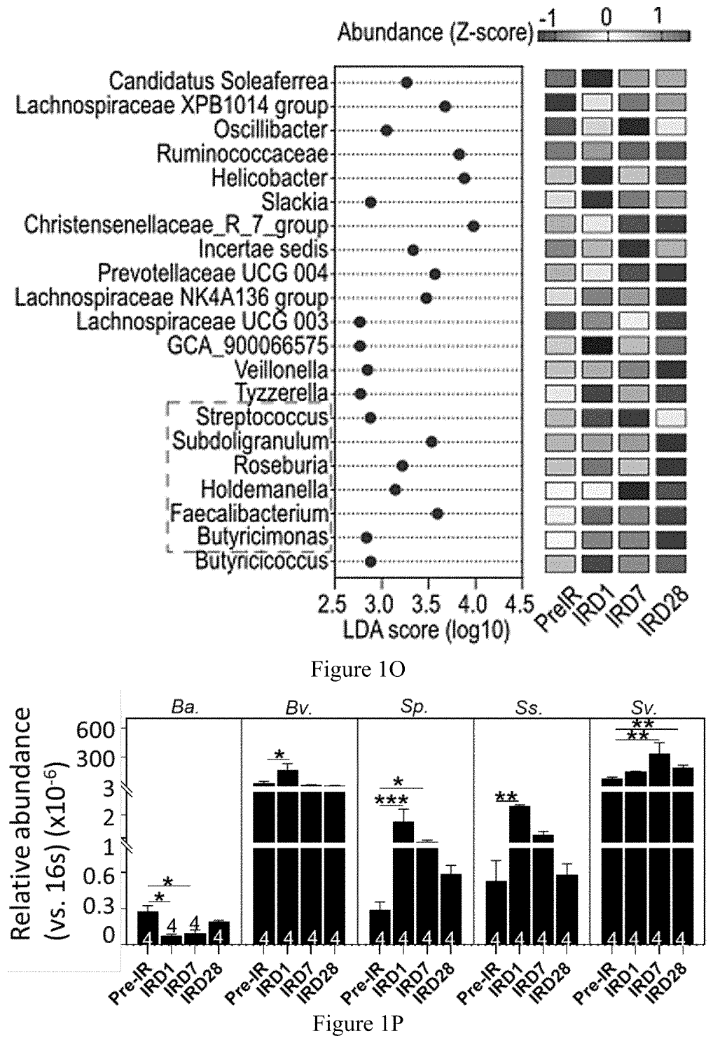

Patient recruitment: The control cases and patients with confirmed ST-elevation myocardial infarction (STEMI) were recruited from National Cheng Kung University Hospital (NCKUH), China Medical University Hospital (CMUH) and Far Eastern Memorial Hospital (FEMH) (FIG. 1). All participants were over 20 years of age. Patients coronary angiogram-confirmed STEMI for the first time were recruited. Participants with kidney and liver diseases were excluded. For control cases, the stool and plasma were collected once. For STEMI cases, stools and plasma were collected at two time points: within three days of percutaneous coronary intervention (PCI) (STEMIT1) and again approximately 28 days after PCI (STEMIT2). The recruitment and sample collection were approved by the IRB of collaborating hospitals as well as Academia Sinica.

16S rRNA NGS: Stool DNA was purified with the innuPREP Stool DNA Kit (Analytik Jena). The V3-V4 region of the 16S rRNA gene was amplified by a specific primer set (319F: 5′-CCTACGGGNGGCWGCAG-3′, SEQ ID NO: 29, 806R: 5′-GACTACHVGGGTATCTAATCC-3′, SEQ ID NO: 30) according to the 16S Metagenomic Sequencing Library Preparation procedure (Illumina). In brief, 12.5 ng of gDNA was used for the PCR reaction carried out with KAPA HiFi HotStart ReadyMix (Roche) under the PCR condition: 95° C. for 3 minutes; 25 cycles of: 95° C. for 30 seconds, 55° C. for 30 seconds, 72° C. for 30 seconds; 72° C. for 5 minutes and hold at 4° C. The PCR products with a bright main strip around 500 bp on a 1.5% agarose gel were purified through the AMPure XP beads for the following library preparation. The 16S rRNA V3-V4 region PCR amplicon was subjected to a secondary PCR along with Nextera XT Index Kit with dual indices and Illumina sequencing adapters (Illumina). The indexed PCR product quality was assessed on the Qubit 4.0 Fluorometer (Thermo Scientific) and Qsep100™ system. Equal amount of the indexed PCR product was mixed to generate the sequencing library. Finally, the library was sequenced on an Illumina MiSeq platform and paired 200-bp reads were generated.

Sequencing analysis: After barcode removal, the PCR amplicons were assembled with FLASH (v1.2.11; http://ccb.jhu.edu/software/FLASH/) to create Raw Tags. The assembly was with minimum overlap of 10 base pairs and 0.1 error rate. The Raw Tags were then processed with QIIME 2 (version 2020.11) to create Clean Tags. Chimeric sequences were removed with UCHIME (http://www.drive5.com/usearch/manual/uchime_algo.html) to create Effective Tags for further analysis. The Effective Tags were grouped into Operational Taxonomic Units (OTUs) and classified with UPARSE algorithm RDP Classifier (v2.2; https://rdp.cme.msu.edu/). The database used includes PyNAST (v1.2), GreenGenes (gg_13_8; default), Silva (v138; 2019.12) and NCBI. Diversities of the bacterial community were analyzed with QIIME 2 (version 2020.11).

### Example 1 Recruitment of Patients with Confirmed ST-Elevation Myocardial Infarction (STEMI)

To elucidate the role of gut microbiota on the clinical outcome of myocardial infarction, the patients with ST-elevation myocardial infarction (STEMI) were recruited in collaboration with Dr. Yen-Wen Wu at Far Eastern Memorial Hospital (FEMH, Northern Taiwan), Dr. Kuan-Cheng Chang at China Medical University Hospital (CMUH, Middle Taiwan), and Yen-Wen Liu at National Cheng Kung University Hospital (NCKU, Southern Taiwan) (Table 1). The disease status of the STEMI patients was all confirmed with Electrocardiography (ECG/EKG), echocardiography and catheterization. So far, we have recruited 147 participants in total, including 70 control cases and 77 STEMI cases. Over 60% of the STEMI cases recruited were male. Moreover, the majority of the STEMI cases were recruited from Southern Taiwan; nearly 70% of the STEMI cases were recruited from CKU. Hypertension, hyperlipidemia and diabetes are all comorbidities and risks for cardiovascular diseases. From the STEMI recruitment, we observed that hyperlipidemia contributed more to the STEMI cases recruited. In addition, the body mass index (BMI) did not show any significant difference between the control and STEMI cases. Our data suggest a geographic and gender difference in the incidence of STEMI in Taiwan.

TABLE 1
STEMI patient recruitment. Control cases and patients with confirmed STEMI were
recruited from Far Eastern Memorial Hospital (Northern Taiwan), China Medical University
Hospital (Middle Taiwan), and National Cheng Kung University Hospital (Southern Taiwan).

| | | No. (%) | | |
|---|---|---|---|---|
| | Total (n = | Control (n = | STEMI (n = | |
| Characteristic | 147) | 70) | 77) | P Value |
| Age, mean (SE), y | 54.0 (1.0) | 52.0 (1.4) | 55.7 (1.2) | 0.052 |
| Male | 115 (78.2) | 43 (61.4) | 72 (93.5) | <0.0001 |
| Location | | | | |
| Northern Taiwan | 26 (17.7) | 7 (10.0) | 19 (24.7) | |
| Middle Taiwan | 48 (32.6) | 42 (60.0) | 6 (7.8) | |
| Southern Taiwan | 73 (49.7) | 21 (30.0) | 42 (54.5) | |
| Comorbidities and risk factors | | | | |
| Hypertension | 46 (31.3) | 24 (34.3) | 23 (29.9) | 0.57 |
| Hyperlipidemia | 77 (52.4) | 25 (35.7) | 52 (67.5) | <0.0001 |
| Diabetes | 29 (19.7) | 14 (20.0) | 15 (19.5) | 0.94 |
| Presenting characteristics | | | | |
| Body mass index | | | | |
| Underweight (< 18.5) | 1 (0.7) | 1 (1-4) | 0 (0.0) | |
| Normal (18.5 to <25) | 48 (32.7) | 20 (28.6) | 28 (36.4) | 0.35 |
| Overweight (25 to 30) | 60 (40.8) | 23 (32.8) | 37 (48.1) | 0.15 |
| Obese (30) | 17 (11.6) | 4 (5.7) | 13 (16.9) | 0.63 |

### Example 2 Increment Butyrate-Producing Bacteria in Post-Cardiac Injury Gut Microbial Community

In order to investigate the relationship between gut microbiome and myocardial infarction (MI), we performed 16s V3-V4 NGS on a total of 214 stool samples from n=77 ST-elevation myocardial infarction (STEMI) patients (confirmed with both electrocardiograph and angiography) and n=70 age and BMI-matched controls (Ctrl) (FIG. 1A). Fecal samples from STEMI patients were collected at two time points; within three days of percutaneous coronary intervention (PCI) (STEMIT1) and again approximately 28 days after PCI (STEMIT2). STEMI and Ctrl samples significantly differed in multivariate analysis. The STEMIT1 group held distinct operational taxonomic units (OTUs) from Ctrl and STEMIT2 groups with over 70% of the bacteria found being shared by both sexes (FIG. 1B). Higher α-diversity was observed in STEMIT1 than STEMIT2 group (Shannon's index, accounting for both abundance and evenness of species), regardless of sex (FIG. 1C). Compared with the Ctrl group, both α-diversity and β-diversity (unweighted UniFrac distance) were higher in the STEMIT1 group, representing a more diverse microbiota in this group (FIGS. 1C and 1D). We observed a two-fold increase of Firmicutes/Bacteroidetes ratio in the STEMIT1 group compared with both Ctrl and STEMIT2 groups (FIG. 1E). Moreover, the left ventricle ejection fraction (EF) of STEMI patients was inversely correlated with the Shannon's index and Pielou's evenness (FIG. 1F). To further identify the affected gut microbes, we performed LEfSe (Linear discriminant analysis (LDA) Effect Size) analysis on genus level with the 16s V3-V4 NGS results (Segata et al., 2011, Genome Biol 12, R60. 10. 1186/gb-2011-12-6-r60). Whereas commensal bacteria Bifidobacterium were reduced in STEMIT1 samples, butyrate-producers were more abundant in STEMIT1 samples, including Anaerotruncus, Alistipes, Butyricimonas, Enterococcus, Holdemanella and Subdoligranulum (Mallott and Amato, 2022, Mol Biol Evol 39. 10. 1093/molbev/msab279; Vital et al., 2013, Microbiome 1, 8. 10.1186/2049-2618-1-8) (FIG. 1G). A deep scale metagenome shotgun analysis of sixteen representative samples (five Ctrl and eleven STEMIT1 samples) extended LEfSe at the species level, showing constriction of Bifidobacterium alolescentis and Bifidobacterium ruminantium, and expansion of Butyricimonas virosa, Streptococcus parasanguinis and Streptococcus salivarius in STEMIT1 samples (FIG. 1H). Changes of these bacteria were further validated with species-specific primer sets using quantitative PCR (FIG. 11, Table 2). In order to minimize dietary and environmental influences, we investigated the relationship between gut microbiota and cardiac injury in nonhuman primates which share significant genetic, physiological biochemical and metabolic similarities with humans. Rhesus macaques were subjected to cardiac ischemia for 90 minutes followed by reperfusion (IR model). Stool samples from −1D, D1, D7 and D28 were then examined to profile changes to the gut microbiota (FIG. 1J). The gut microbiota compositions were distinct at each time point (FIG. 1K). Moreover, both α- and β-diversities increased after IR and peaked at IRD28 and IRD7, respectively (FIGS. 1L and 1M). Similar to STEMIT1 samples, Firmicutes/Bacteroidetes ratio was upregulated at IRD1 (FIG. 1N). Butyrate-producing bacteria like Butyricimonas, Faecalibacterium, Holdemanella, Roseburia and Subdoligranulum were enriched after injury (FIG. 10). Comparable to human samples, we also confirmed reduction of Bifidobacterium alolescentis, and enrichment of Butyricimonas virosa, Streptococcus parasanguinis, Streptococcus salivarius and Subdoligranulum variabile after IR using qPCR (FIG. 1P). Conserved increases of butyrate-producing bacteria after cardiac injury in both human and nonhuman primates indicates that butyrate producers may influence post-injury cardiac repair.

TABLE 2
Primer sets for bacterial validation.

| | Forward (F) or | | |
|---|---|---|---|
| | Reverse (R) | | |
| Bacteria | primer | Sequence (5′-3′) | SEQ NO |
| Anaerotruncus colihominis | (F) | CTAAAACAGAGGGCGGCGAC | 1 |
| DMA 17241 | (R) | CTTCGGGTGTTACCCGGACTC | 2 |
| Clostridium symbiosaum | (F) | AACTGGAGTGTCGGAGAGGT | 3 |
| ATCC 14940 | (R) | TTCATCGTTTACGGCGTGGA | 4 |
| Coprococcus comes | (F) | GGCGTGTAATGACGCTTTT | 5 |
| ATCC 27758 | (R) | AGTCTCTCCAGAGTGCCCAT | 6 |
| Ruminococcus torques | (F) | CGAGGTGGAGCAAATCCCAA | 7 |
| ATCC 27756 | (R) | ACTGACTTCGGGCGTTACTG | 8 |
| Bacteroides caccae | (F) | ATGGGGAAACCCATACGCC | 9 |
| ATCC 43185 | (R) | CCAGAGTCCTCAGCATGACC | 10 |
| Bacteroides thetaiotaomicron | (F) | GGGCAGTGATCTACGTGTCAAG | 11 |
| VPI-5482 | (R) | CTGCATCGTACCCAAAATCGTCTG | 12 |
| Providencia stuartii | (F) | TCCCTAGAGGAGTGGCTTCC | 13 |
| ATCC 29914 | (R) | CTCCCGAAGGCACTAAAGCA | 14 |
| Collinsella aerofaciens | (F) | CTCTCCGGAGGGAAGCGAG | 15 |
| ATCC 25986 | (R) | TGTCTCAGTCCCAATCTGGC | 16 |
| Bifidobacterium adolescentis | (F) | CCGGTGTAACGGTGGAATGT | 17 |
| ATCC 15703 | (R) | GACACGGAGACCGTGGAATG | 18 |
| Bifidobacterium ruminantium | (F) | TCCTATCAGGTAGTCGGCGG | 19 |
| | (R) | GCTTGCTCCCAGTCAAAAGC | 20 |
| Streptococcus parasanguinis | (F) | ATGGGGTGACCATCGCAAAA | 21 |
| ATCC 15912 | (R) | GAGTCAAAACCGTTGCGGTC | 22 |
| Streptococcus salivarius | (F) | GTTATGAGCTCAGGCTCGCT | 23 |
| | (R) | GCAGCAATTCCGCCTTCTTT | 24 |
| Butyricimonas virosa | (F) | AAGGATGACGAGTCATTCGATGC | 25 |
| JCM 15149 | (R) | CTTCACTTGTTCCGCCTCCC | 26 |
| Subdoligranulum variabile | (F) | GATCCGCCATCGGATGAGG | 27 |
| | (R) | GTGCAATATCCCCCACTGCT | 28 |

### Example 3 Machine Learning Strategy for Gut Microbiome-Driven STEMI Diagnosis

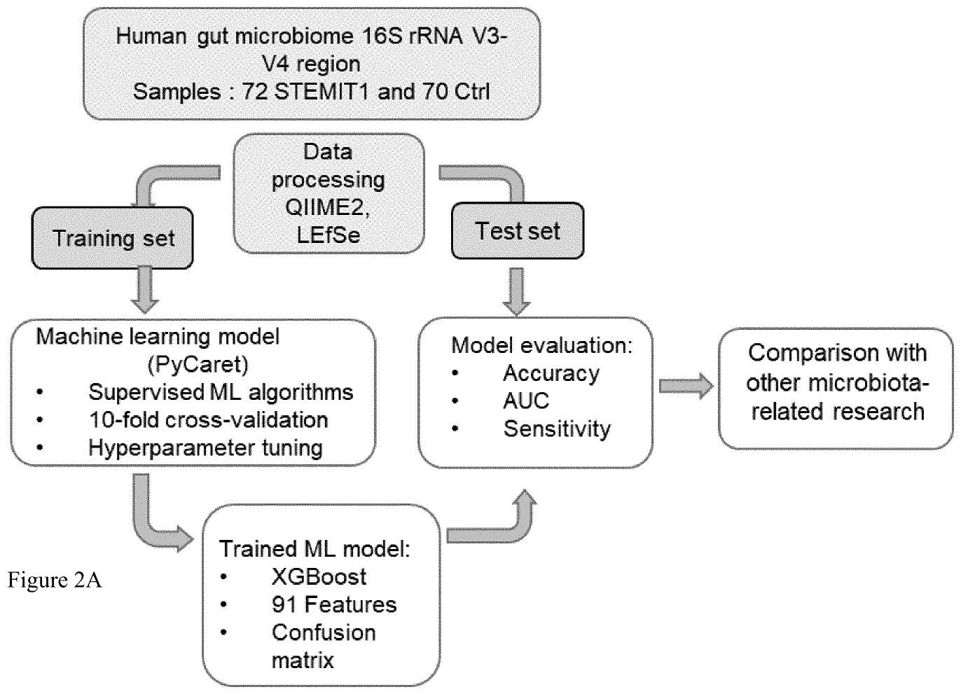

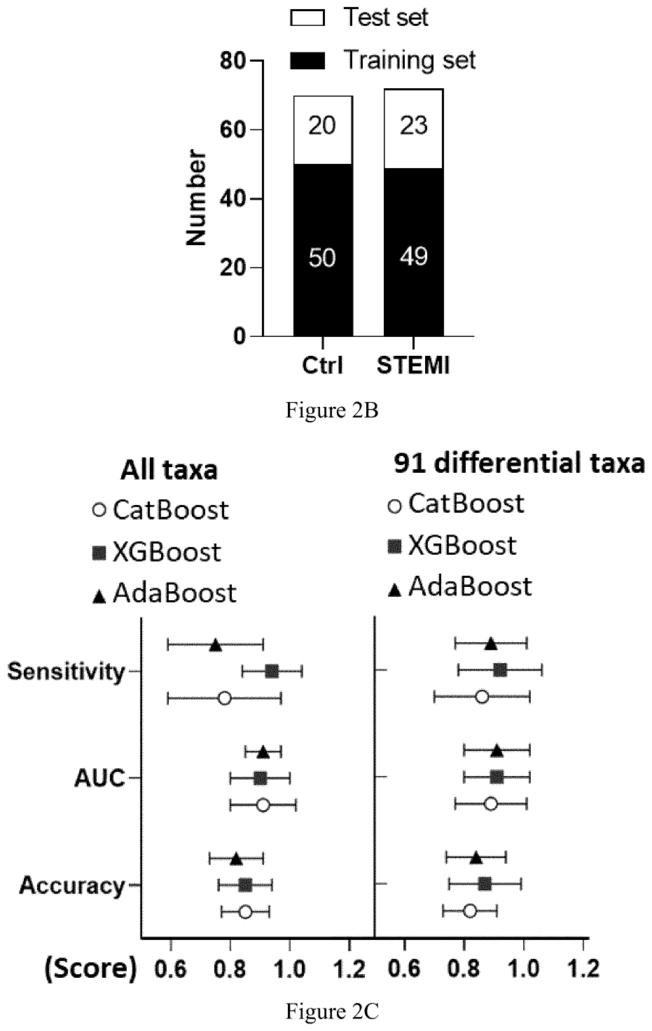

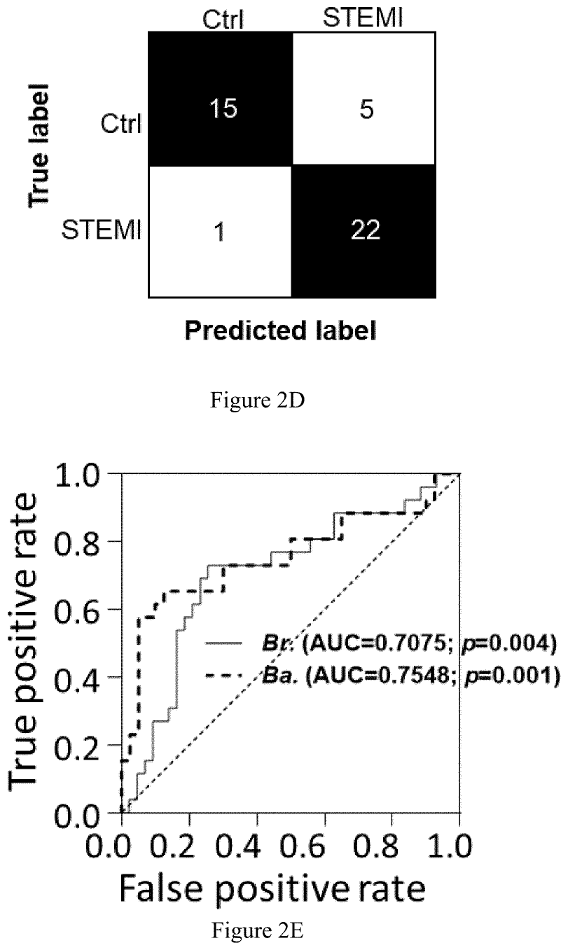

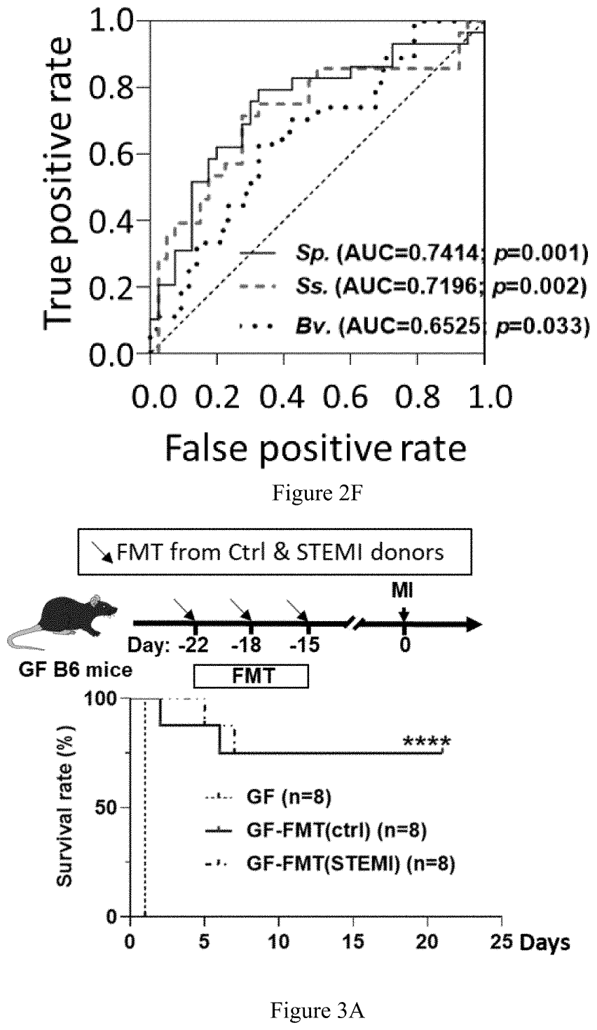

To investigate the possibility of using a gut microbial composition as a prediction tool for STEMI, we performed four types of supervised machine learning classifiers on the features of bacterial taxa using PyCaret package (Ai et al., 2017, Oncotarget 8, 9546-9556. 10.18632/oncotarget.14488) (FIG. 2A). By means of random data splitting, 70% of the samples were used as a training set to build the prediction model, while the remaining 30% were assigned as a test set to evaluate the model performance (FIG. 2B). To begin with, we trained the prediction models with all taxa, which showed an AUC of 0.85 (FIG. 2C). To have a model with comparable performance but a much smaller number of features for economic and clinical application consideration, we then trained the models with 91 highly differential taxonomic features selected from LEfSe (LDA core>2.0). Accordingly, 91 taxa represented the most variable features to perform consistently in various machine learning models (FIG. 2C). Analyzing the test set with XGBoost using 91 taxonomic features resulted in 86.1% accuracy and 96% sensitivity, indicating the potential for precise determination of STEMI patients using a gut microbiota configuration (FIG. 2D). Remarkably, the prediction power of our model is higher than reported machine learning studies in predicting diseases using gut microbiome sequencing data (Table S3). Moreover, receiver operating characteristic (ROC) curve analysis showed an AUC of more than 0.65 for B. alolescentis, B. ruminantium, B. virosa, S. parasanguinis and S. salivarius as identified by shotgun metagenome, suggesting the favorable power of these bacteria for STEMI diagnosis (FIGS. 2E-2F).

### Example 4 STEMI Fecal Microbiome Transplantation (FMT) Deteriorates Post-Injury Cardiac Function in Germ-Free (GF) Mice

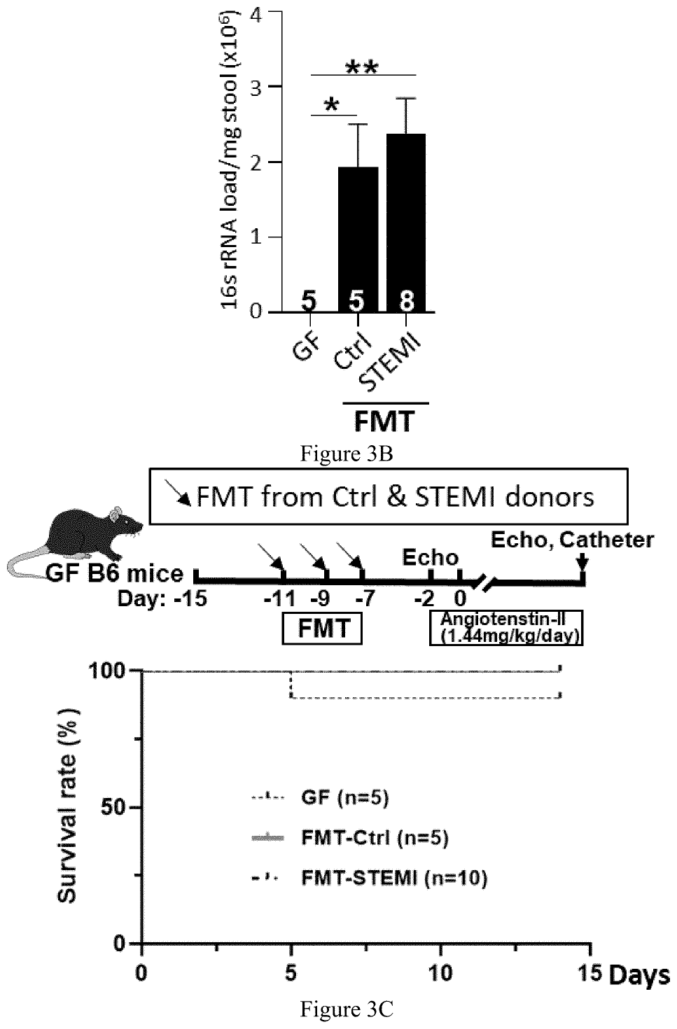

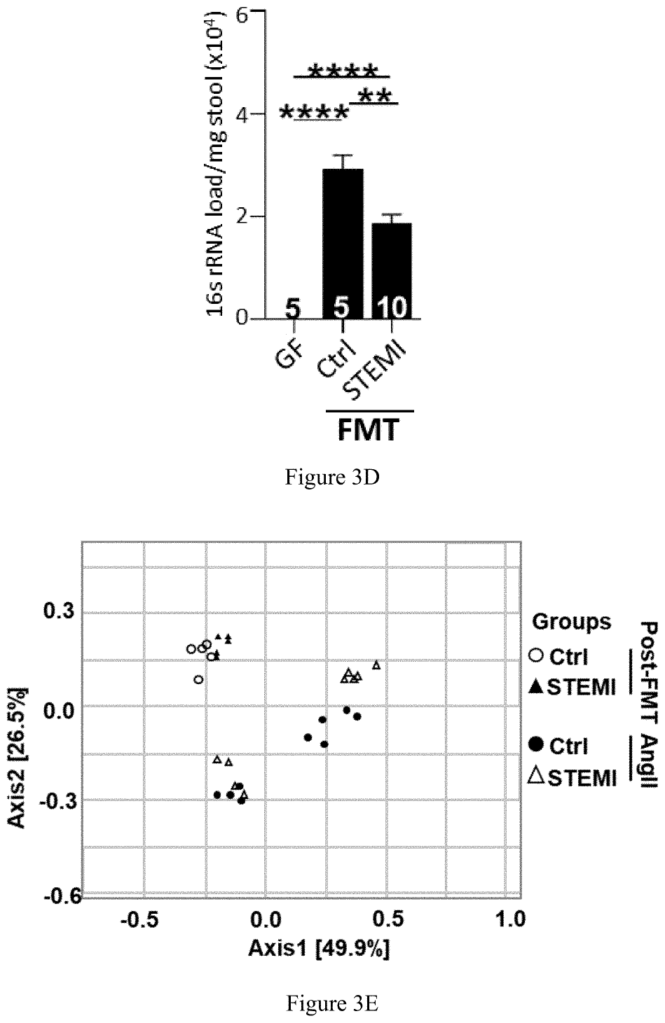

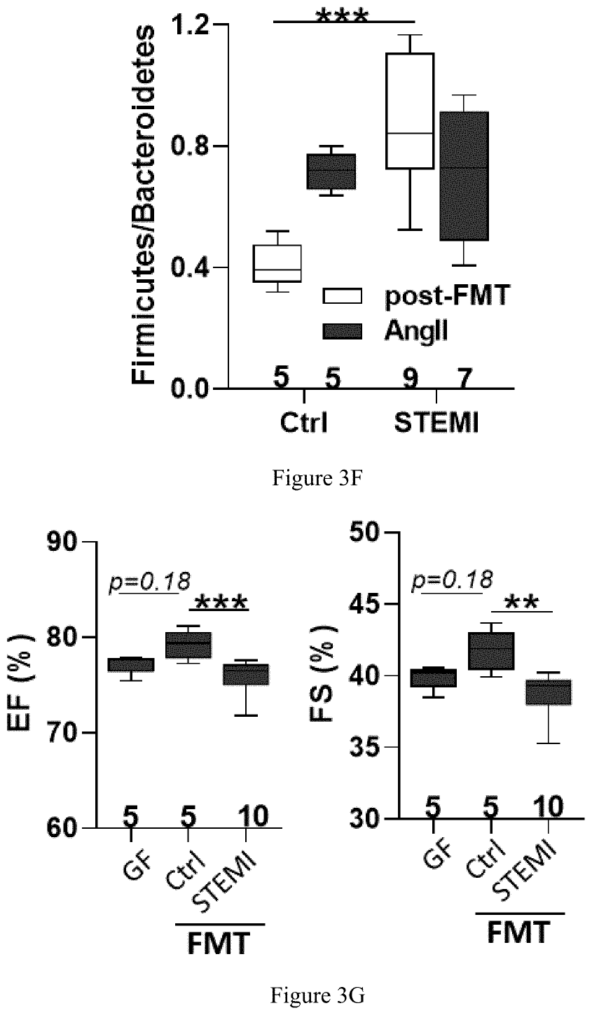

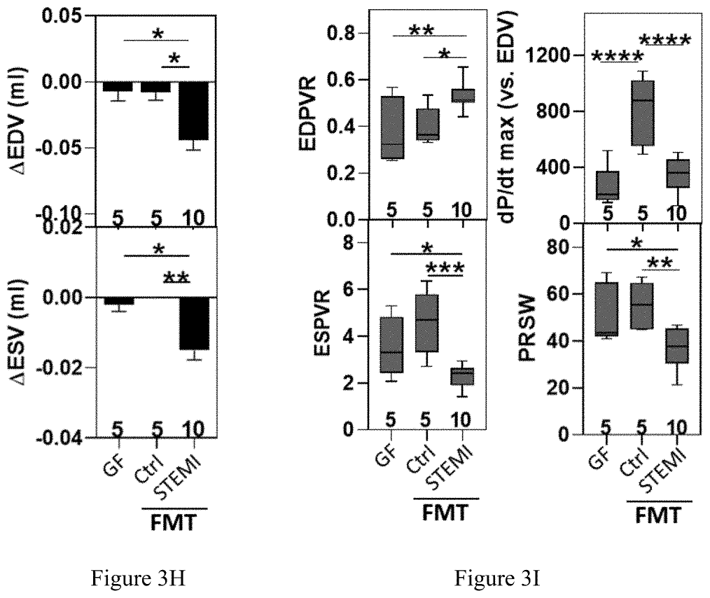

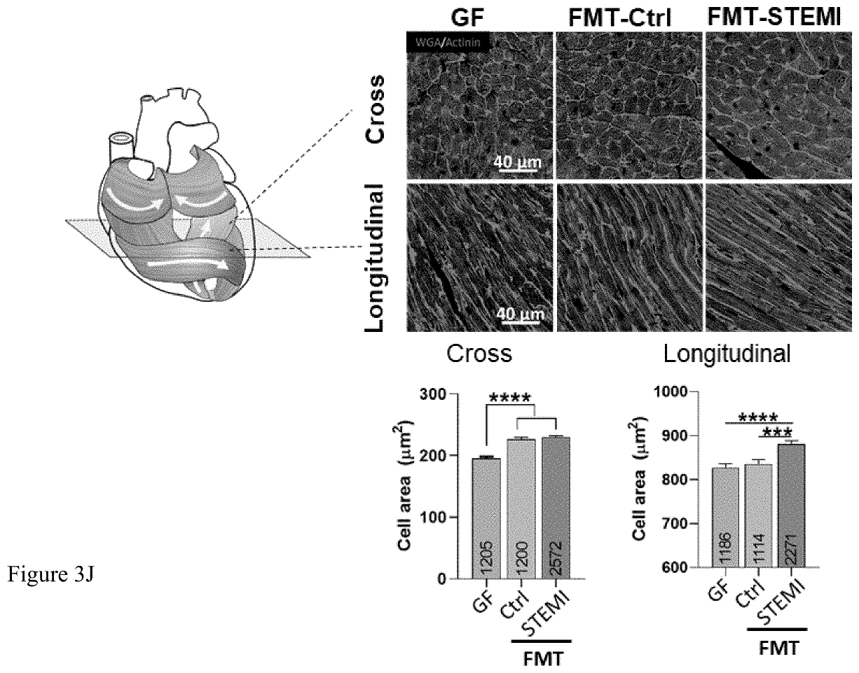

To experimentally assess the influence of the human gut microbiota on post-injury cardiac function, we transplanted human fecal samples from Ctrl and STEMIT1 into the 12-week old male C57BL/6J germ-free (GF) mice (FIGS. 3A and 3B). After challenging the mice with MI surgery via left descending coronary artery ligation, we observed an unexpectedly high mortality rate of the mice which received STEMIT1 FMT (FIG. 3B), suggesting that STEMI gut microbiota contained substances which caused adverse response to MI. Thus, in order to further investigate the effects of STEMI microbiota on post-injury cardiac function, we utilized a milder injury model using angiotensin-II (1.44 mg/kg/day) for fourteen days (FIGS. 3C-3D). Principle coordinates analysis discriminated gut microbiomes from Ctrl- and STEMI-FMT mice (FIG. 3E). Additionally, the Firmicutes/Bacteroidetes ratio was higher in the gut microbiota of STEMI-FMT mice and the ratio was also elevated in Ctrl-FMT mice treated with angiotensin-II (FIG. 3F). Compared with Ctrl-FMT, the STEMI-FMT mice showed worsened left ventricle ejection fraction (EF) and fraction shortening (FS) as well as reduced end diastolic and systolic left ventricle volume (EDV and ESV) (FIGS. 3G and 3H). Likewise, STEMI-FMT increased stiffness and compromised contractility of the left ventricle of recipient mice, showing higher EDPVR as well as ESPVR, PRSW and dP/dt max (vs. EDV) (FIG. 3I). This would be attributed to cardiomyocyte hypertrophy (FIG. 3J). Moreover, STEMI-FMT mice had thinner submucosal and shorter villi in the intestine, parallel to profound circulating pro-inflammatory cytokines such as IL-17 and IL-22, even before angiotensin-II challenge in STEMI-FMT mice.

### Example 5 Enrichment of Ketone Body Metabolism was Enriched During Post-Injury Cardiac Repair

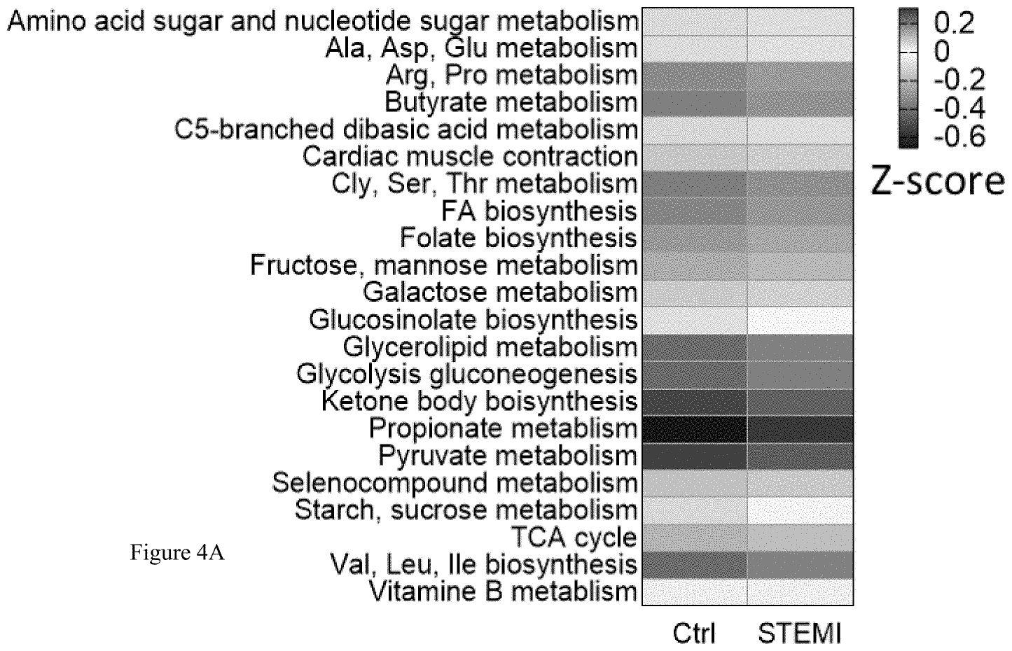

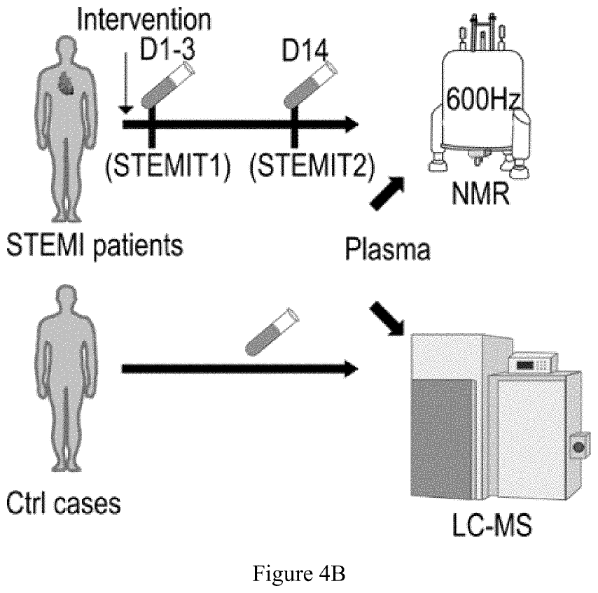

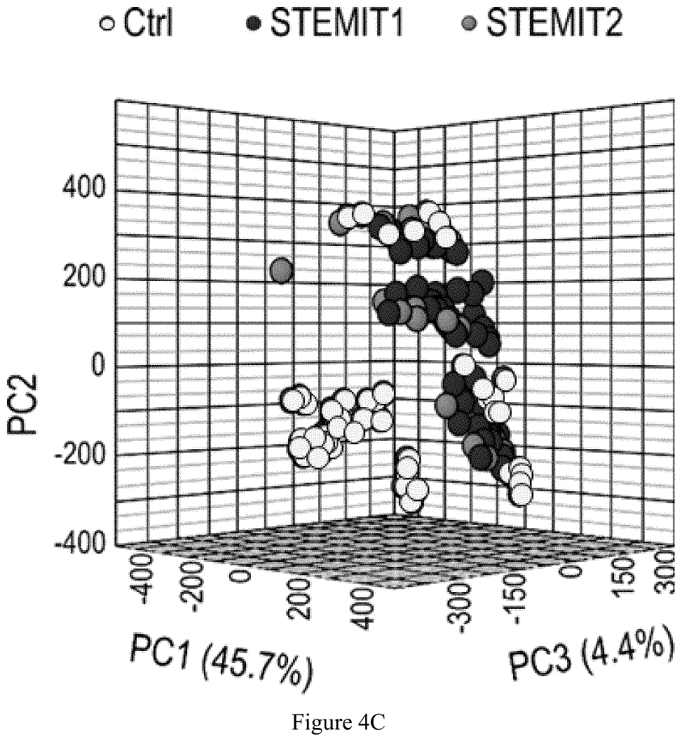

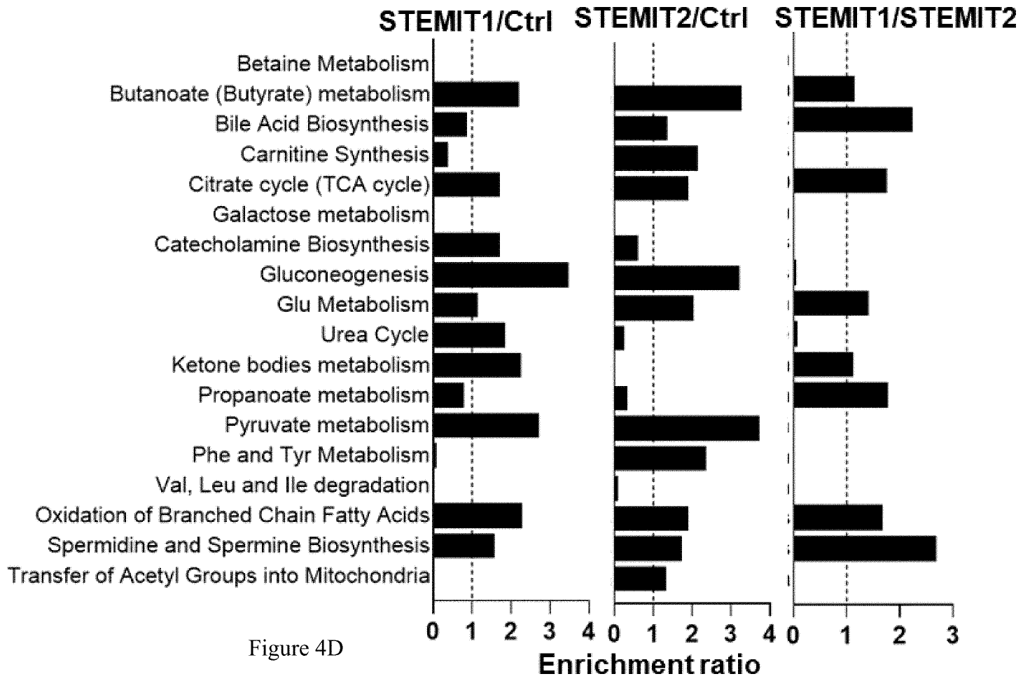

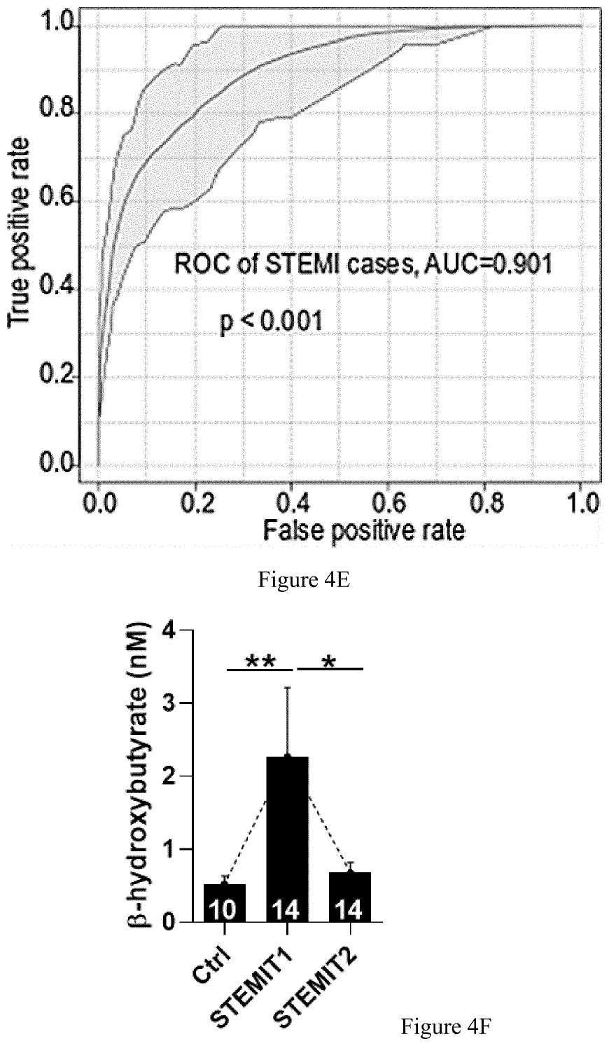

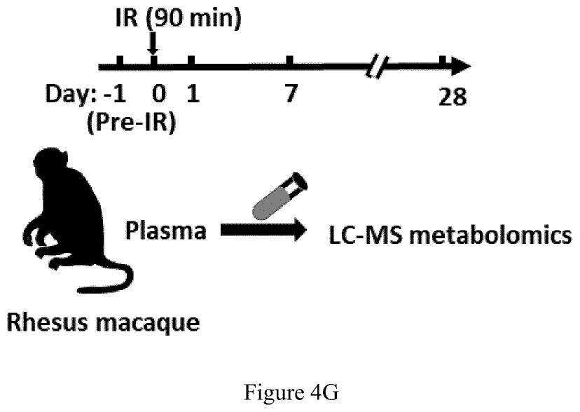

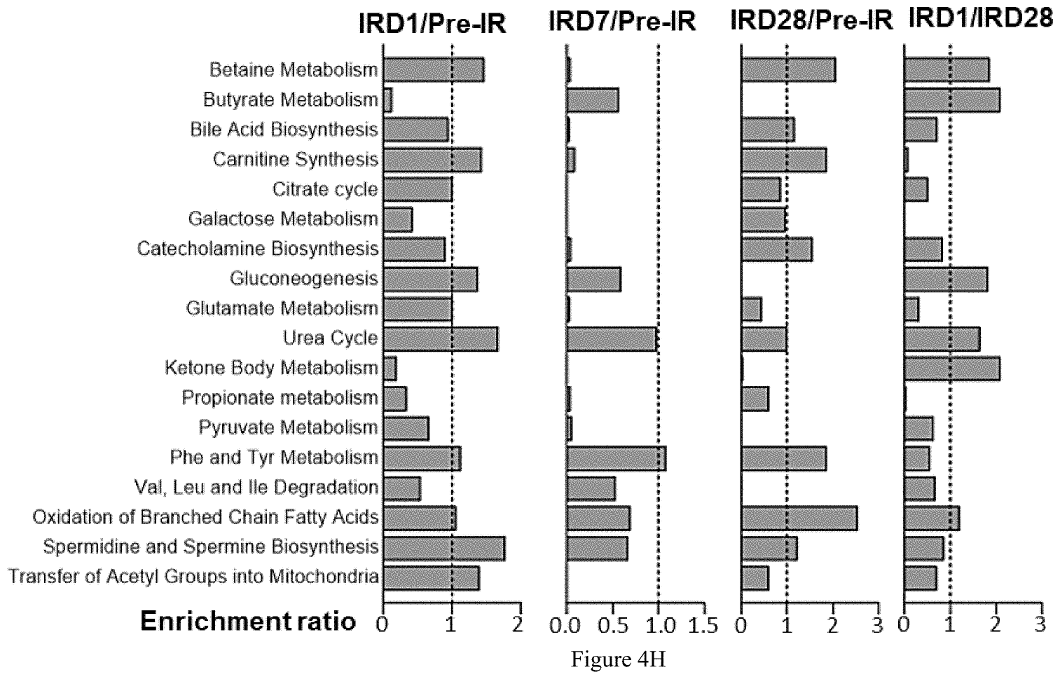

The gut microbiomes may influence the host through bacterially-produced small molecules (Kasahara et al., 2018, Nat Microbiol 3, 1461-1471. 10.1038/s41564-018-0272-x; Yachida, et al., 2019, Nat Med 25, 968-976. 10.1038/s41591-019-0458-7). Shotgun metagenomics analysis of the Ctrl and STEMIT1 stools revealed STEMIT1-associated enrichment of bacterial genes involved in metabolism of amino acids, short chain fatty acids and the TCA cycle (FIG. 4A). Increased bacterial genes of butyrate metabolism coincided with accumulation of butyrate-producing bacterial in STEMIT1 samples (FIG. 4A). Of note, bacterial genes for ketone body metabolism were also augmented in the STEMIT1 samples (FIG. 4A). To determine the changes of plasma metabolites in STEMI samples, we performed both NMR and LC-MS metabolomics (FIG. 4B). Initial large scale NMR metabolomics screening revealed distinct clustering of Ctrl, STEMIT1 and STEMIT2 metabolites (FIG. 4C). Metabolic pathway analysis from LC-MS metabolomics showed enrichment of fatty acid, amino acid and ketone body metabolism as well as biosynthesis of polyamines in STEMIT1 plasma compared with both Ctrl and STEMIT2 plasma, highlighting drastic metabolic alterations in the acute phase of MI (FIG. 4D). Compared with Ctrl, metabolisms of butyrate, glutamate and pyruvate, and biosynthesis of bile acid, carnitine and spermidine/spermine were persistently enriched in the plasma of both STEMIT1 and STEMIT2, suggesting that these were STEMI-associated metabolic changes (FIG. 4D). ROC curves based on the enrichment of metabolites in ketone body metabolism showed AUC of about 0.901, suggesting alteration of ketone body metabolism as potential prediction marker for STEMI (FIG. 4E). Increased β-hydroxybutyrate in STEMI plasma was further validated with a colorimetric assay (FIG. 4F). We next addressed whether similar metabolism alteration could be observed in the nonhuman primate IR model (FIG. 4G). Similar to human samples, metabolic alteration began early in the acute phase after IR, showing enrichment of nearly all metabolic pathways examined at IRD1 compared with PreIR and IRD28 (FIG. 4H). Unlike human plasma, enrichment of butyrate, pyruvate and glutamate metabolism was only observed on IRD1 and IRD7 (FIG. 4H). Moreover, colorimetric assay validated that plasma β-hydroxybutyrate increased at IRD1 and then reduced over time (FIG. 41). Together these data highlight the importance of butyrate and ketone body metabolism after cardiac injury.

### Example 6 Butyrate Supplementation Confers Better Post-MI Cardiac Function Preservation in Intact Gut Microbiota

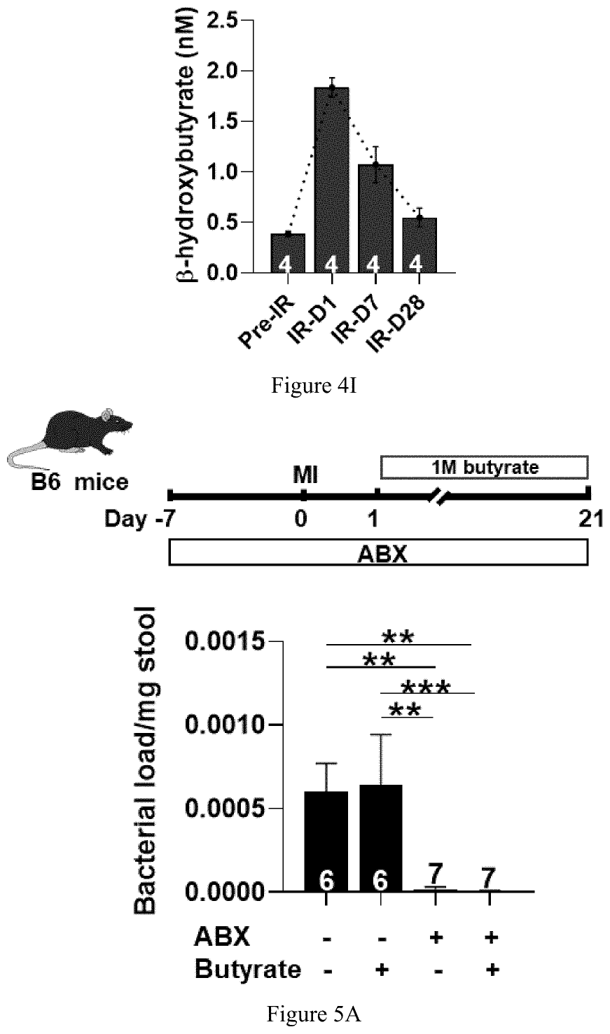

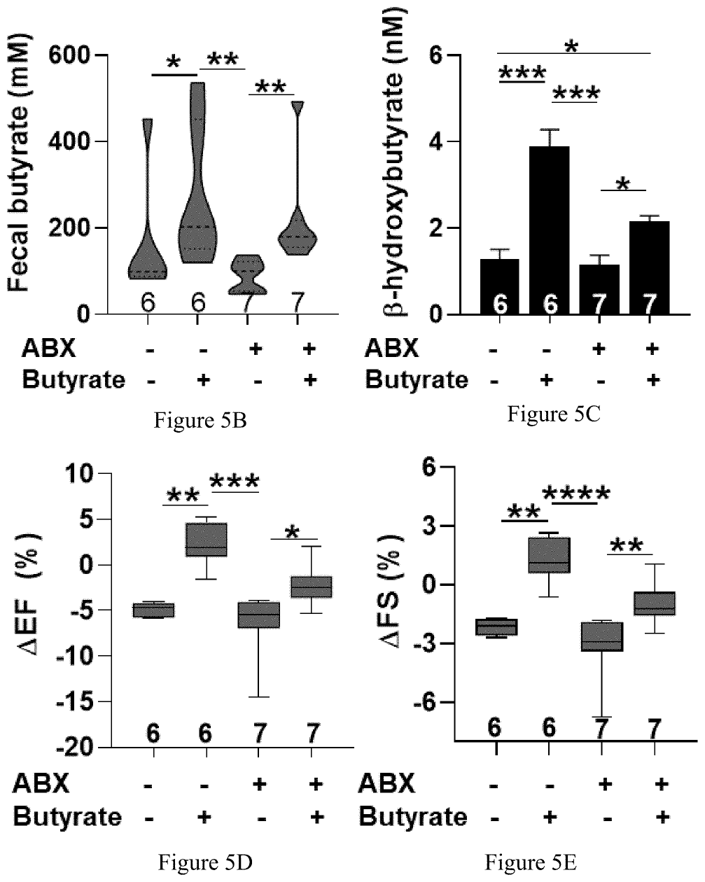

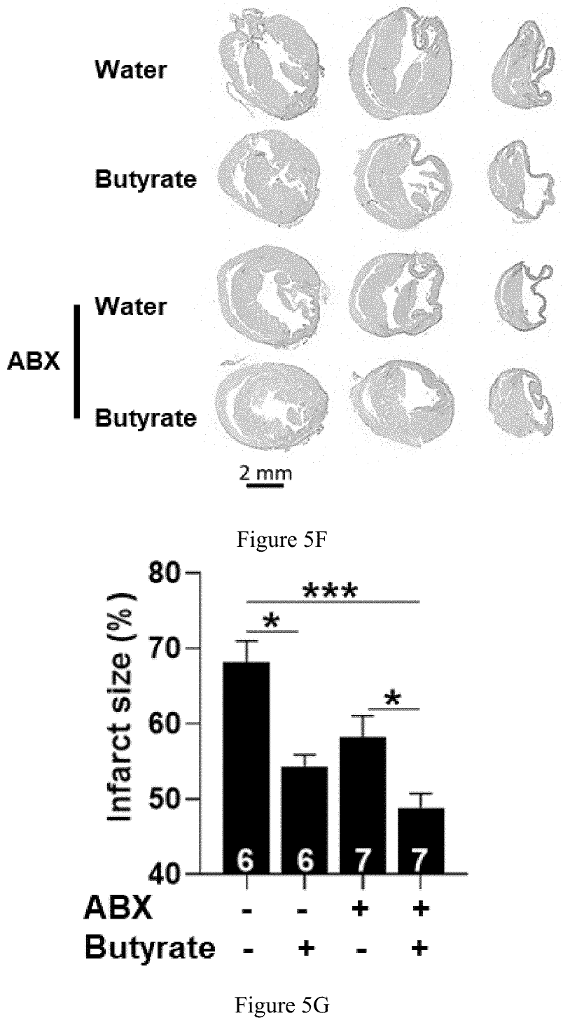

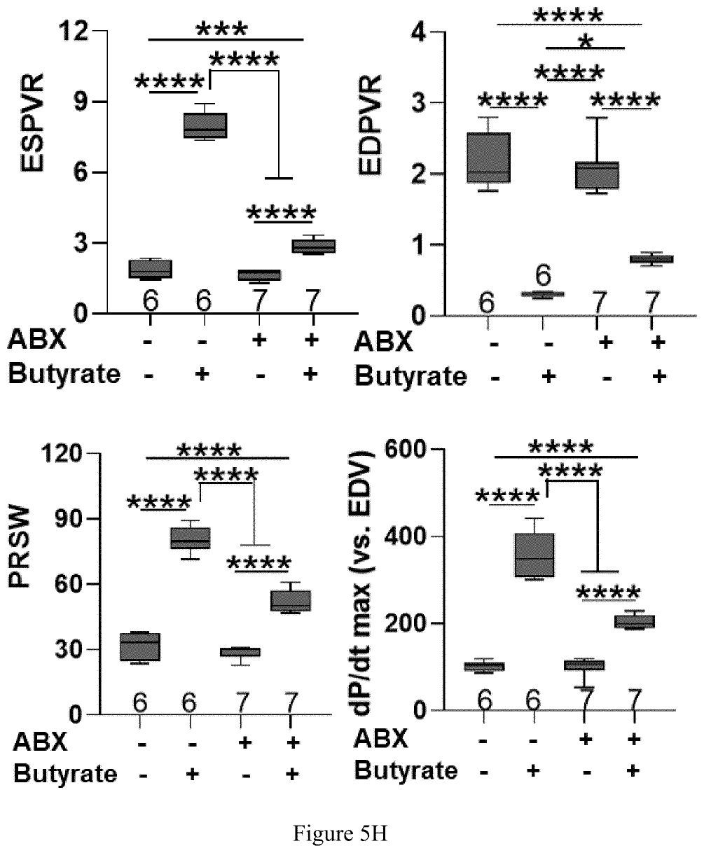

From metabogenomic analysis, we identified an increase of butyrate-producing bacteria after cardiac injury in both humans and nonhuman primates. In order to determine the influence of butyrate on post-MI cardiac repair, we used a combination of broad spectrum antibiotics (ABX) to deplete the host microbiome, and supplemented C57BL/6J mice with butyrate via gavage beginning one day after MI and continuing for twenty days (FIG. 5A). Supplementation successfully increased the levels of fecal butyrate plasma β-hydroxybutyrate (FIGS. 5B and 5C). Interestingly, plasma level of β-hydroxybutyrate was even higher when host gut microbiota was intact (FIG. 5C). Improved EF and FS were noticed in the butyrate-supplemented groups, especially those with intact gut microbiota (FIGS. 5D and 5E). Supplementing mice with butyrate reduced the post-MI infarct size (FIGS. 5F and 5G). Additionally, cardiac mechanical properties were improved in mice receiving butyrate, in particular in the context of intact gut microbiota, presenting higher ESPVR, PRSW and dP/dt max (vs. EDV) as well as lower EDPVR (FIG. 5H). Together these data illustrate the importance of intact gut microbiota for the protective role of butyrate during post-MI cardiac repair.

### Example 7 Colonization of Butyrate-Producing Bacteria Ameliorates Post-MI Cardiac Injury

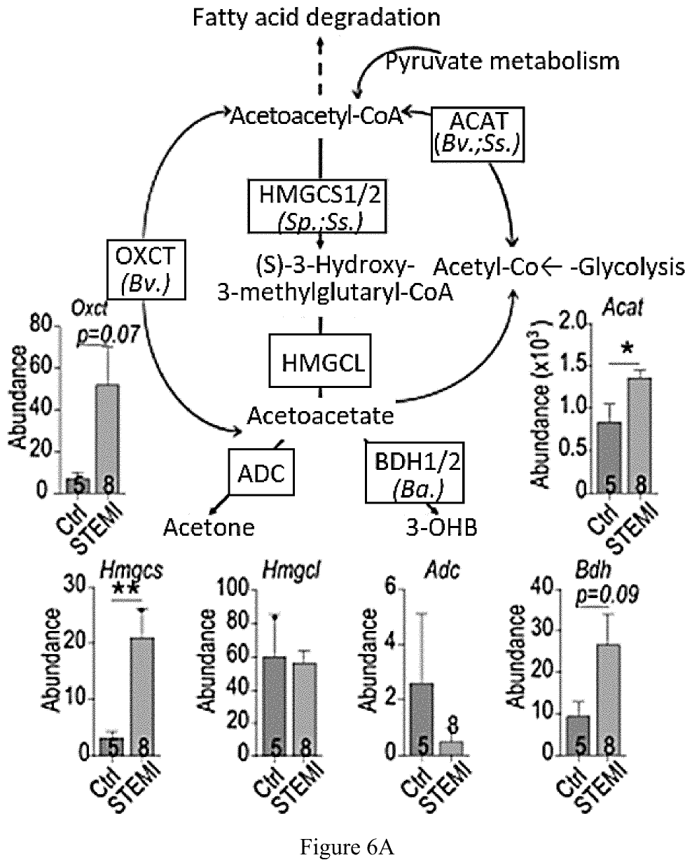

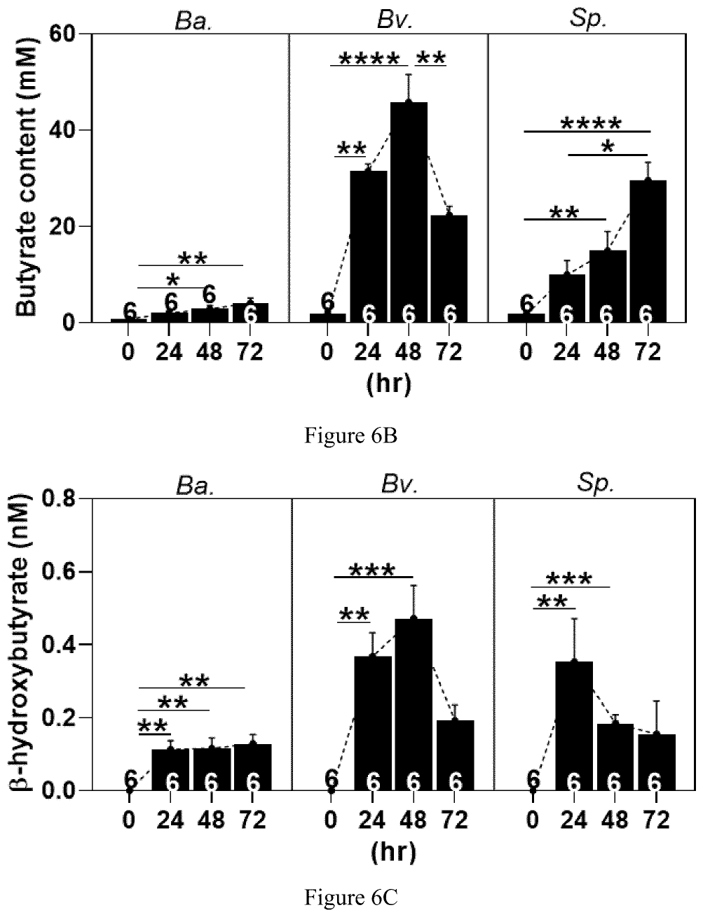

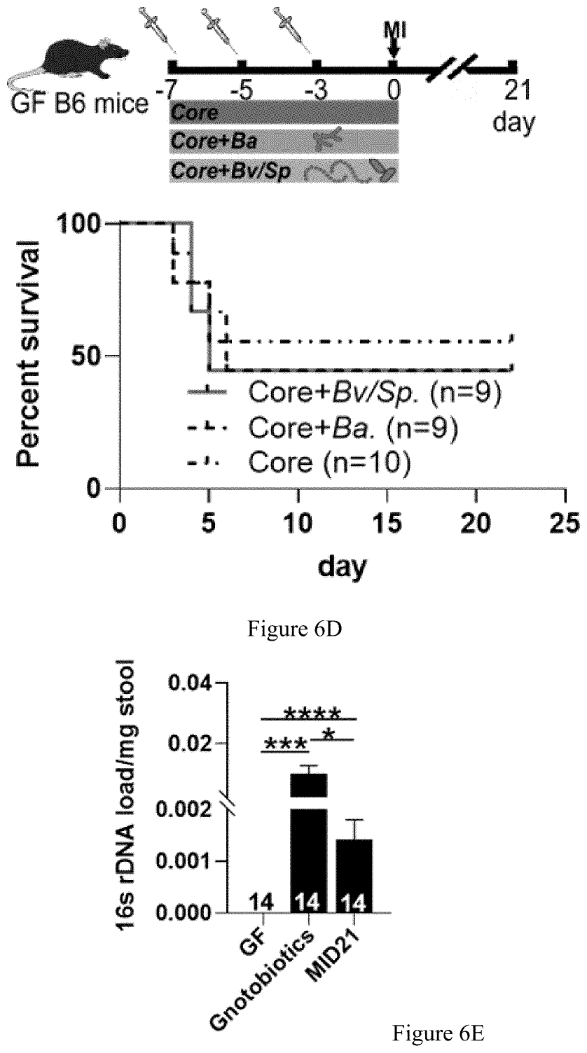

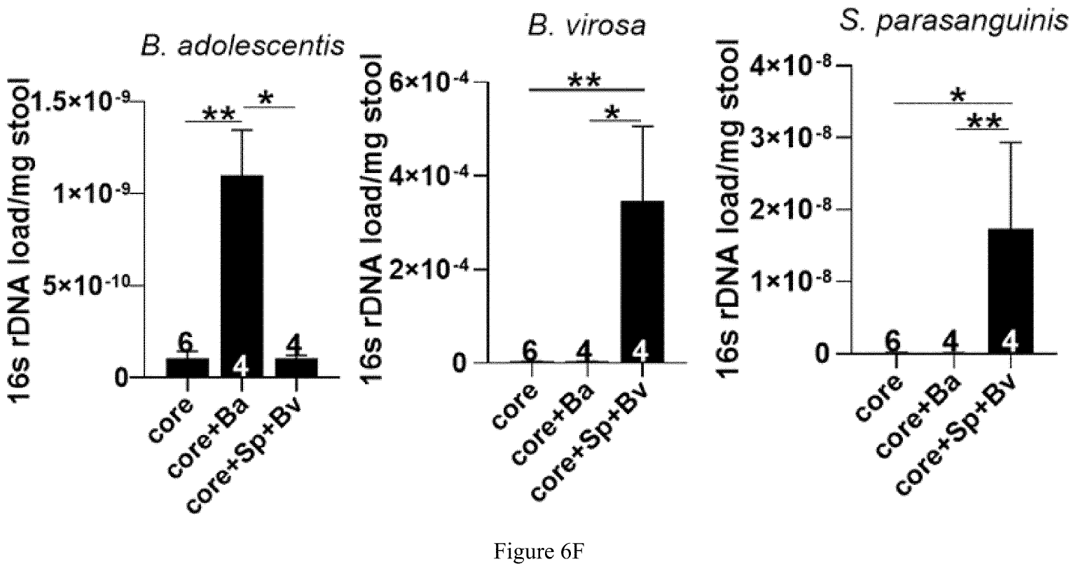

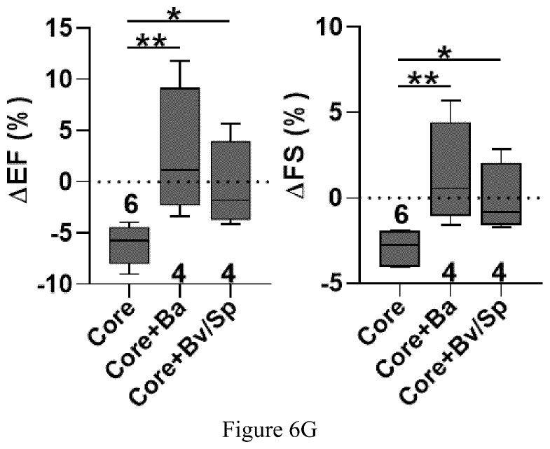

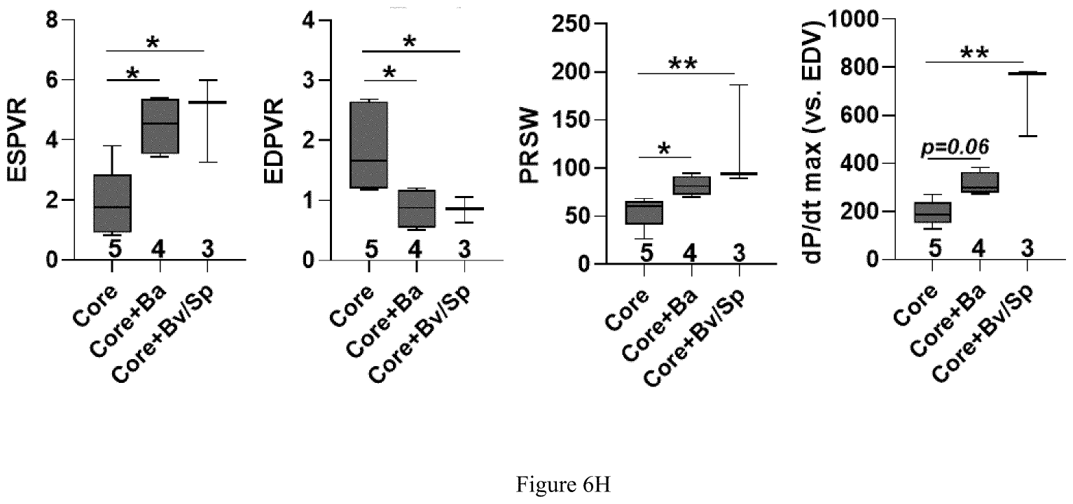

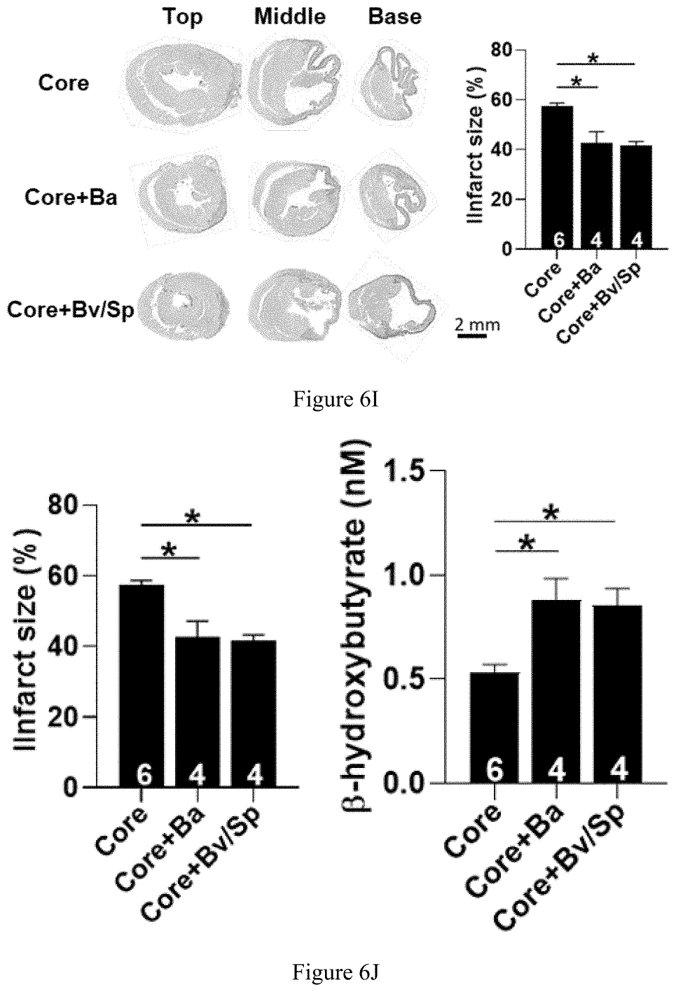

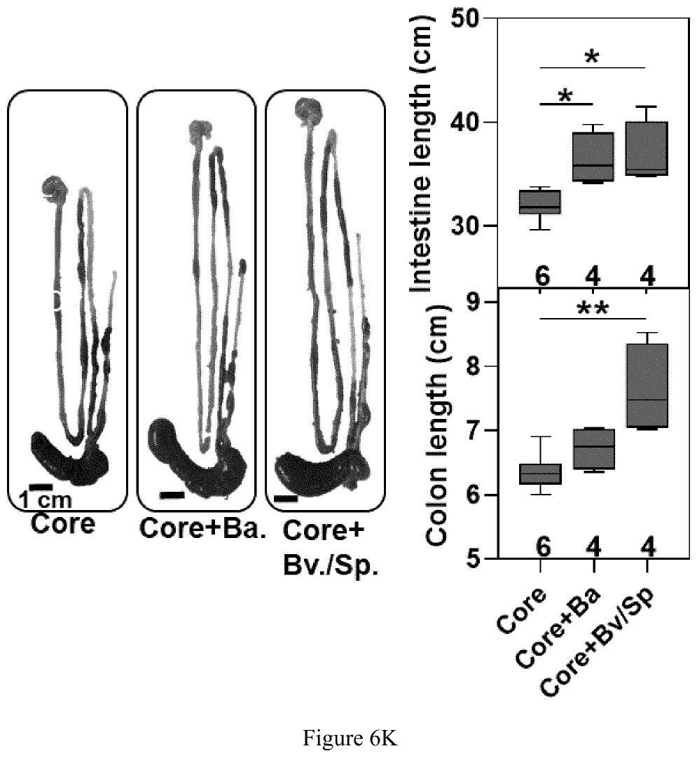

The shotgun metagenomics showed that bacterial genes (ACAT, HMGCS, OXCT and BDH) encoding key-step enzymes for ketone body metabolisms were enriched in the STEMI samples, and the bacteria of note include STEMIT1 associated B. adolescentis, B. virosa and S. parasanguinis (FIG. 6A). B. adolescentis, B. virosa and S. parasanguinis were capable of producing butyrate in culture, except that B. adolescentis was less potent (FIG. 6B). Concomitantly, all three bacteria also produced β-hydroxybutyrate (FIG. 6C). To examine whether B. adolescentis, B. virosa and S. parasanguinis gnotobiotics could influence post-injury cardiac function in GF mice, we inoculated GF mice with a “core” community that included eight species commonly found in human gut microbiota with a limited butyrate-producing capability (Anaerotruncus colihominis, Bacteroides caccae, Bacteroides thetaiotaomicron, Clostridium symbiosum, Collinsella aerofaciens, Coprococcus comes, Providencia stuartii and Ruminococcus torques) (Kasahara et al., 2018, Nat Microbial 3, 1461-1471. 10.1038/s41564-018-0272-x); or a combination of core with Ctrl-predominant B. adolescentis (Core+ Ba), or STEMI-associated B. virosa/S. parasanguinis (Core+Bv/Sp) prior to MI surgery, and observed the mice for twenty-one days after MI induction (FIGS. 6D-6F). Post-MI survival rates were similar for mice from all three groups (FIG. 6D). We confirmed successful colonization with Ba or Bv/Sp (FIG. 6F) and these mice showed improved cardiac function compared to mice receiving core bacteria only. Mice transplanted with B. adolescentis, or B. virosa/S. parasanguinis, showed positive changes in EF and FS (FIG. 6G). Higher ESPVR, PRSW and dP/dt max (vs. EDV) as well as lower EDPVR were discovered in B. adolescentis, and B. virosa/S. parasanguinis gnotobiotic mice, revealing improved preservation of cardiac mechanical properties (FIG. 6H). Moreover, the average infarct size after twenty-one days was smaller in both B. adolescentis, and B. virosa/S. parasanguinis gnotobiotic mice (FIG. 6I). These mice also showed an increase in the plasma level of β-hydroxybutyrate (FIG. 6J). Additionally, B. virosa/S. parasanguinis gnotobiotic mice displayed a longer intestine and colon than those inoculated with core bacteria only (FIG. 6K). These data demonstrate the cardiac protective role of B. adolesenctis and B. virosa/S. parasanguinis and also indicate the contribution of bacteria-associated ketone body metabolism in post-MI cardiac protection.

While the present invention has been described in conjunction with specific embodiments set forth above, many alternatives thereto and modifications and variations thereof will be apparent to those of ordinary skill in the art. All such alternatives, modifications and variations are regarded as falling within the scope of the present invention.

What is claimed is:

1. A method for early detecting or diagnosing the likelihood of myocardial infarction (MI), predicting prognosis or treatment outcome, and/or differentiating MI in a subject, wherein the method comprises:
obtaining a sample comprising gut microbiota of the subject;
performing genetic analysis on the gut microbiota; and
determining the likelihood of MI as an indicator of detection or diagnosis of MI or prediction of prognosis or treatment outcome of MI if:
(i) decreased abundance of gut microbial genera Bacteroidetes, and/or Bifidobacterium;
(ii) increased abundance of gut microbial genera Streptococcus, Clostridium and/or Butyricimonas;
(iii) increased abundance ratio of gut microbial genera Firmicutes/Bacteroidetes; and/or
(iv) increased complexity and diversity of gut microbial genera;
in the sample is found when compared to a normal control.

2. The method according to claim 1, wherein the sample comprises a tissue of the gut or a fecal sample.

3. The method according to claim 1, wherein the method further comprises analyzing 16S rRNA of the metagenome of the gut microbiota.

4. The method according to claim 1, wherein the determination of (i) comprises determining the abundance of the gut microbial bacteria Bifidobacterium adolescentis and/or Bifidobacterium ruminantium.

5. The method according to claim 1, wherein the determination of (ii) comprises determining the abundance of the gut microbial bacteria Butyricimonas virosa, Clostridium asparagiforme, Streptococcus parasanguinis and/or Streptococcus salivarius.

6. The method according to claim 1, wherein the determination of (i), (ii), (iii) and/or (iv) comprises analyzing V3-V4 region of the 16S rRNA of the metagenome of the gut microbiota.

7. The method according to claim 1, wherein the determination of (i), (ii), (iii) and/or (iv) comprises performing next generation sequencing of 16S rRNA of the metagenome of the gut microbiota.

8. The method according to claim 1, wherein the determination of (i), (ii), (iii) and/or (iv) comprises performing next generation sequenceing of 16S rRNA of the metagenome of the gut microbiota, shotgun metagenomic sequencing the gut microbiota, linear discriminant analysis (LDA) on the gut microbiota, or effect size (LEfSe) analysis on the gut microbiota.

9. The method according to claim 1, wherein the determination of (iv) comprises performing alpha or beta diversity calculating for determining the complexity and diversity of the gut microbiota.

10. The method according to claim 1, wherein the normal control is obtained by:
obtaining a group of control samples comprising gut microbiota of a group of normal subjects; and
performing genetic analysis on the gut microbiota of the group of normal subjects, wherein the genetic analysis on the gut microbiota of the group of normal subjects is the same as that of the subject.

11. The method according to claim 1, wherein the MI is ST-elevation myocardial infarction (STEMI).

12. A biomarker for the early diagnosis, prognosis and differentiation of myocardial infarction in a subject, wherein the biomarker is selected from one or more gut microbial genera Bacteroidetes, Bifidobacterium, Streptococcus, Clostridium, Butyricimonas, Firmicutes and Bacteroidetes.

13. The biomarker according to claim 12, which is selected from the group consisting of:
a combination of gut microbial genera Bacteroidetes, and/or Bifidobacterium;
a combination of gut microbial genera Streptococcus, Clostridium and/or Butyricimonas; and
a combination of gut microbial genera Firmicutes and Bacteroidetes.

14. A kit for the early diagnosis, prognosis, recurrence and differentiation of myocardial infarction in a subject, wherein the kit comprises a detecting molecule for detecting the biomarker according to claim 12.

15. The kit according to claim 14, wherein the detecting molecule is for analyzing 16S rRNA of the metagenome of the gut microbiota.

16. The kit according to claim 14, wherein the kit further comprises a group of control samples comprising gut microbiota of a group of normal subjects.

17. A method for treating and/or ameliorating myocardial infarction in a subject, wherein the method comprises colonizing Bifidobacterium adolescentis, Butyricimonas virosa and/or Streptococcus parasanguinis in the gut of the subject.

18. The method according to claim 17, which further comprises colonizing a butyrate-producing bacterium in the gut.

19. The method according to claim 18, wherein the butyrate-producing bacterium is selected from the group consisting of Anaerotruncus colihominis, Bacteroides caccae, Bacteroides thetaiotaomicron, Clostridium symbiosum, Collinsella aerofaciens, Coprococcus comes, Providencia stuartii and Ruminococcus torques.

20. The method according to claim 17, wherein the method is for improving cardiac function, improving preservation of cardiac mechanical properties, and/or decreasing average infarct size.


SEQUENCE LISTING (30 sequences)

SEQ ID NO: 1 - DNA, 20 bp, Artificial Sequence
  synthetic primer
ctaaaacagagggcggcgac

SEQ ID NO: 2 - DNA, 21 bp, Artificial Sequence
  synthetic primer
cttcgggtgttacccggactc

SEQ ID NO: 3 - DNA, 20 bp, Artificial Sequence
  synthetic primer
aactggagtgtcggagaggt

SEQ ID NO: 4 - DNA, 20 bp, Artificial Sequence
  synthetic primer
ttcatcgtttacggcgtgga

SEQ ID NO: 5 - DNA, 20 bp, Artificial Sequence
  synthetic primer
ggcgtgtaatgacgcctttt

SEQ ID NO: 6 - DNA, 20 bp, Artificial Sequence
  synthetic primer
agtctctccagagtgcccat

SEQ ID NO: 7 - DNA, 20 bp, Artificial Sequence
  synthetic primer
cgaggtggagcaaatcccaa

SEQ ID NO: 8 - DNA, 20 bp, Artificial Sequence
  synthetic primer
actgacttcgggcgttactg

SEQ ID NO: 9 - DNA, 19 bp, Artificial Sequence
  synthetic primer
atggggaaacccatacgcc

SEQ ID NO: 10 - DNA, 20 bp, Artificial Sequence
  synthetic primer
ccagagtcctcagcatgacc

SEQ ID NO: 11 - DNA, 22 bp, Artificial Sequence
  synthetic primer
gggcagtgatctacgtgtcaag

SEQ ID NO: 12 - DNA, 24 bp, Artificial Sequence
  synthetic primer
ctgcatcgtacccaaaatcgtctg

SEQ ID NO: 13 - DNA, 20 bp, Artificial Sequence
  synthetic primer
tccctagaggagtggcttcc

SEQ ID NO: 14 - DNA, 20 bp, Artificial Sequence
  synthetic primer
ctcccgaaggcactaaagca

SEQ ID NO: 15 - DNA, 19 bp, Artificial Sequence
  synthetic primer
ctctccggagggaagcgag

SEQ ID NO: 16 - DNA, 20 bp, Artificial Sequence
  synthetic primer
tgtctcagtcccaatctggc

SEQ ID NO: 17 - DNA, 20 bp, Artificial Sequence
  synthetic primer
ccggtgtaacggtggaatgt

SEQ ID NO: 18 - DNA, 20 bp, Artificial Sequence
  synthetic primer
gacacggagaccgtggaatg

SEQ ID NO: 19 - DNA, 20 bp, Artificial Sequence
  synthetic primer
tcctatcaggtagtcggcgg

SEQ ID NO: 20 - DNA, 20 bp, Artificial Sequence
  synthetic primer
gcttgctcccagtcaaaagc

SEQ ID NO: 21 - DNA, 20 bp, Artificial Sequence
  synthetic primer
atggggtgaccatcgcaaaa

SEQ ID NO: 22 - DNA, 20 bp, Artificial Sequence
  synthetic primer
gagtcaaaaccgttgcggtc

SEQ ID NO: 23 - DNA, 20 bp, Artificial Sequence
  synthetic primer
gttatgagctcaggctcgct

SEQ ID NO: 24 - DNA, 20 bp, Artificial Sequence
  synthetic primer
gcagcaattccgccttcttt

SEQ ID NO: 25 - DNA, 23 bp, Artificial Sequence
  synthetic primer
aaggatgacgagtcattcgatgc

SEQ ID NO: 26 - DNA, 20 bp, Artificial Sequence
  synthetic primer
cttcacttgttccgcctccc

SEQ ID NO: 27 - DNA, 20 bp, Artificial Sequence
  synthetic primer
gatccggcatcggattgagg

SEQ ID NO: 28 - DNA, 20 bp, Artificial Sequence
  synthetic primer
gtgcaatatcccccactgct

SEQ ID NO: 29 - DNA, 17 bp, Artificial Sequence
  synthetic primer
  misc_feature (9)..(9): n is a, c, g, or t
cctacgggnggcwgcag

SEQ ID NO: 30 - DNA, 21 bp, Artificial Sequence
  synthetic primer
gactachvgggtatctaatcc